(19) United States
(12) Patent Application Publication
(10) Pub. No.: US 2024/0091260 A1
(43) Pub. Date: Mar. 21, 2024

(54) CHIMERIC ANTIGEN RECEPTORS THAT BIND PREFERENTIALLY EXPRESSED ANTIGEN IN MELANOMA (PRAME)/HLA-A2 TO TREAT CANCER

(71) Applicant: Fred Hutchinson Cancer Center, Seattle, WA (US)

(72) Inventors: Soheil Meshinchi, Seattle, WA (US); Danielle Kirkey, Seattle, WA (US); Anisha Loeb, Seattle, WA (US); Quy H. Le, Puyallup, WA (US)

(73) Assignee: Fred Hutchinson Cancer Center, Seattle, WA (US)

(21) Appl. No.: 18/454,684

(22) Filed: Aug. 23, 2023

Related U.S. Application Data
(60) Provisional application No. 63/373,311, filed on Aug. 23, 2022.

(51) Int. Cl. A61K 35/17; A61K 38/21; A61K 39/00; A61P 35/02; C07K 14/74

(52) U.S. Cl. CPC A61K 35/17; A61K 38/217; A61K 39/4611; A61K 39/4631; A61K 39/464411; A61P 35/02; C07K 14/70539; A61K 2239/15; A61K 2239/21; A61K 2239/22; A61K 2239/25; A61K 2239/48

(57) ABSTRACT

Chimeric antigen receptors (CAR) that bind Preferentially Expressed Antigen in Melanoma (PRAME) ALY(SEQ ID NO: 94)/HLA-A2 are disclosed. The CAR can be used to treat PRAME/HLA-A2 expressing cancers such as the t(8;21), Inv(16), and KMT2A-r forms of acute myeloid leukemia (AML).

### CROSS-REFERENCE TO RELATED APPLICATION

This application claims priority to U.S. Provisional Patent Application No. 63/373,311 filed Aug. 23, 2022, the entire contents of which are incorporated by reference herein.

### REFERENCE TO SEQUENCE LISTING

The Sequence Listing associated with this application is provided in XML format in lieu of a paper copy and is hereby incorporated by reference into the specification. The name of the file containing the Sequence Listing is 2Y91637-Sequence Listing.xml. The file is 124,860 bytes, was created on Aug. 23, 2023, and is being submitted electronically via Patent Center.

### FIELD OF THE DISCLOSURE

The current disclosure provides chimeric antigen receptor (CAR) that bind Preferentially Expressed Antigen in Melanoma (PRAME) ALYVDSLFFL (ALY (SEQ ID NO: 94))/HLA-A*0201 (HLA-A2) to treat PRAME-expressing cancers such as acute myeloid leukemia (AML).

### BACKGROUND OF THE DISCLOSURE

Adoptive transfer of T cells engineered to express chimeric antigen receptors (CAR) has achieved impressive outcomes in the treatment of refractory/relapsed B cell acute lymphoblastic leukemia (B-ALL), providing potentially curative options for these patients (Gardner et al., Blood. 2017, 129(25):3322-3331; Maude et al., N Engl J Med. 2016 Mar. 10, 374(10):998]. N Engl J Med. 2014, 371(16):1507-1517; and Turtle et al., J Clin Invest. 2016, 126(6):2123-2138). The use of CAR T cell therapy in AML, however, is still in its infancy with limitations due to the innate heterogeneity associated with AML and lack of AML-specific targets for therapeutic development.

### SUMMARY OF THE DISCLOSURE

The current disclosure provides chimeric antigen receptors (CAR) that bind Preferentially Expressed Antigen in Melanoma (PRAME) ALYVDSLFFL (ALY, SEQ ID NO: 94)/HLA-A*0201 (HLA-A2) to treat PRAME-expressing cancers. In particular embodiments, the CAR include a binding domain derived from the Pr20 monoclonal antibody. CAR T cells targeting the PRAME antigen in AML (referred to as PRAME _{mTCR}CAR T cells) disclosed herein demonstrate in vitro and in vivo efficacy against HLA-A2 restricted AML cells expressing the PRAME antigen, providing a novel approach to target PRAME with CAR T cells. Particular embodiments utilize CAR disclosed herein to treat t(8;21) AML, Inv(16) AML, or KMT2A-r AML. Particular embodiments include co-administering interferon gamma (IFNγ) to increase PRAME expression by cancer cells.

### BRIEF DESCRIPTION OF THE FIGURES

Some of the drawings submitted herein may be better understood in color. The patent or application file contains at least one drawing executed in color. Copies of this patent or patent application publication with color drawing(s) will be provided by the Office upon request and payment of the necessary fee.

FIGS. 1A-1C. PRAME transcript expression in AML. (FIG. 1A) (FIG. 1B) Waterfall plot showing PRAME expression (TPM) in pediatric AML cohort (TpAML) compared to normal bone marrow (NBM) and peripheral blood (PB) CD34+ samples (FIG. 1A) and in adult SWOG AML cohort (FIG. 1B). (FIG. 1C) Waterfall plot showing PRAME expression (TPM) across AML subtypes in TpAML cohort.

FIGS. 2A-2E. PRAME expression related to age and outcome (related to FIGS. 1A-1D). (FIG. 2A) PRAME mRNA transcript expression (expressed as log 2 TPM) for various age groups. Statistical analysis by Wilcoxon rank sum tests. (FIG. 2B) PRAME expression for various CNV (x-axis labels: —Y; CBL deletion; del5q; monosomy7; trisomy21; trisomy8; trisomy8/trisomy21; Unknown; No Relevant CNV; CD34+PB; and NBM) and (FIG. 2C) known fusions in AML (expressed as log 2 TPM) (x-axis labels: CBFA2T3-GLIS2; CBFB-MYH11; DEK-NUP214; ETV6-MNX1; FUS-ERG; KAT6A-CREBBP; KMT2A-X; None; NUP98-KDM5A; NUP98-NSD1; RBM15-MKL1; RUNX1-CBFA2T3; RUNX1-RUNX1T1; Other AML; CD34+PB; NBM). (FIG. 2D) Kaplan-Meier Curves of Disease-free survival and (FIG. 2E) overall survival for PRAME-positive patients (above 5 TPM) and PRAME-negative patients (below 5 TPM).

FIG. 3. Pr20 antigen expression in cell lines and primary AML blasts. Top, flow cytometric analysis of Pr20 binding (red) in cell lines expressing both PRAME and HLA-A2 (THP-1, OCI-AML2, and RS4;11 HLA-A2+) or expressing only PRAME (K562 and parental RS4;11) or HLA-A2 (MV4;11 HLA-A2+) compared to isotype control (grey). (y-axis labels: 0, 20, 40, 60, 80, 100; x-axis labels: PE-A, 10¹, 10², 10³, 10⁴, 10⁵). Bottom, expression of HLA-A2 (blue) compared to isotype control (grey) for the indicated cell lines. (y-axis labels: 0, 20, 40, 60, 80, 100; x-axis labels: APC-A, 10¹, 10², 10³, 10⁴, 10⁵). Data representative of three independent experiments.

FIG. 4. Flow cytometric analysis of Pr20 antigen expression in three patient index cases.

FIGS. 5A-5D. PRAME ^{mTCR}CAR T cells demonstrate in vitro efficacy against Pr20-positive AML cells. (FIG. 5A) Diagram of PRAME ^{mTCR}CAR construct. SP=GM-CSFR signal peptide; TM=transmembrane domain; CD=costimulatory domain; SD=stimulatory domain; tCD19=truncated CD19. (FIG. 5B) Cytolytic activity of CD8 T cells unmodified or transduced with PRAME ^{Mtcr}CAR construct after 24 hours coculture with THP-1, OCI-AML2, and K562 cells. Data presented are mean specific lysis±SD from 3 technical replicates at indicated E:T ratios. (FIG. 5C) Cytolytic activity of CD8 T cells unmodified or transduced with PRAME ^{Mtcr}CAR construct after 24 hours coculture with primary AML blasts. Data presented are mean specific lysis±SD from 3 technical replicates at indicated E:T ratios. (FIG. 5D) Concentration of secreted IL-2, IFN-γ, and TNF-α in the supernatant following 24 hour of coculture with CD4 or CD8 T cells at 1:1 E:T ratio as measured by enzyme-linked immunosorbent assay. Where concentrations of cytokines are too low to discern, the number above the x-axis indicates the average concentration. Statistical significance was determined by unpaired Student t test, assuming unequal variances. *P<0.05, **P<0.005, ***P<0.0005. Data are representative of 2 donors.

FIGS. 6A-6C. Reactivity of PRAME ^{mTCR}CAR T cells against Pr20-positive AML cells. (Related to FIGS. 5A-5D) (FIG. 6A) Cytolytic activity of unmodified and PRAME ^{mTCR}CAR CD8 T cells derived from an additional donor was assayed against AML target cells as described in FIGS. 3A, 3B. Data presented are mean leukemia specific lysis+/−standard deviation from 3 technical replicates at indicated E:T ratios and timepoints. (FIGS. 6B, 6C) Concentration of secreted IL-2, IFN-gamma, and TNF-alpha in the supernatant following 24 hour of co-culture with CD4 or CD8 T cells at 1:1 E:T ratio as measured by ELISA. Where concentrations of cytokines are too low to discern, the number above the x-axis indicates the average concentration.

FIGS. 7A-7D. PRAME ^{mTCR}CAR T cells eliminate PR20-positive AML cells in vivo. (FIG. 7A) Bioluminescent imaging of OCI-AML2, THP1, and K562-bearing mice treated with unmodified or PRAME ^{mTCR}CAR T-cells 5×10⁶ T cells per mouse. Shown are representative images at the indicated timepoints. (FIG. 7B) Disease burden measured by radiance from OCI-AML2, THP1, and K562 xenograft mice treated with unmodified or PRAME ^{mTCR}CAR T-cells. Shown is the average radiance and SEM. N=5 mice per group for OCI-AML2 and THP-1 models and N=3 for K562. (FIG. 7C) T cell expansion of unmodified and PRAME ^{mTCR}CAR T cells 6 days after T cell infusion from OCI-AML2, THP1 and K562 xenograft mice (FIG. 7D). Kaplan-Meier survival curves of OCI-AML2, THP1, and K562 xenograft mice treated with unmodified or PRAME ^{mTCR}CAR T cells. Statistical differences in survival were evaluated using Log-rank Mantel-Cox. n.s., not significant.

FIGS. 8A-8C. IFN-gamma treatment enhances Pr20 antigen and HLA-A2 expression and increase the cytolytic activity of PRAME ^{mTCR}CAR T cells. (7A, 7B). Flow cytometric analysis of Pr20 antigen (FIG. 8A) and HLA-A2 (FIG. 8B) expression in OCI-AML2, THP-1 and K562 treated with DMSO (blue) or IFN-gamma (10 ng/mL, orange) for three days. Data representative of three independent experiments. (y-axis labels: 0, 20, 40, 60, 80, 100; x-axis labels: 10¹, 10², 10³, 10⁴, 10⁵). (FIG. 8C). Cytolytic activity of unmodified or PRAME ^{mTCR}CAR CD8 T cells co-cultured for 16 hours with THP1, OCI AML2 and K562 cells after pre-treatment with IFN-gamma (10 ng/mL) or DMSO control. Data presented are mean specific lysis+/−SD from 3 technical replicates at indicated E:T ratios. Solid blue and red lines represent IFN-gamma treatment versus dashed lines representing untreated controls.

FIG. 9. Truncated CD19 expression in human CD4 and CD8 T cell subsets in the mouse peripheral blood harvested from OCI-AML2 xenografts at day 6 post T cell injection. (Related to FIGS. 7A-7D) Quantification of percent truncated CD19 positive cells amongst CD4 and CD8 cells in OCI-AML2 and THP-1 xenografts treated with unmodified or PRAME ^{mTCR}CAR T cells. Data presented are the average+/−standard deviation from 5 mice.

FIGS. 10A, 10B. IFN-gamma enhanced Pr20 antigen and HLA-A2 expression in OCI-AML2 and increased the cytolytic activity of PRAME ^{mTCR}CAR T cells. (related to FIGS. 8A-8C). (FIG. 10A) Flow cytometric analysis of Pr20 antigen (left) and HLA-A2 (right) expression in OCI-AML2 treated with DMSO (blue) or IFN-gamma (10 ng/mL, orange) for three days. (y-axis labels: Normalized to Mode, 0, 20, 40, 60, 80, 100; x-axis labels: (left panel) PE-A, 10¹, 10², 10³, 10⁴, 10⁵; (right panel) APC-A, 10¹, 10², 10³, 10⁴, 10⁵). (FIG. 10B) Cytolytic activity of unmodified or PRAME ^{mTCR}CAR CD8 T cells co-cultured for 6 hours with OCI-AML2 after pre-treatment with IFN-gamma (10 ng/mL) or DMSO control. Data presented are mean specific lysis+/−SD from 3 technical replicates as indicated 5:1 E:T ratio.

FIG. 11. Schematic of genetic construct encoding a PRAME CAR.

FIG. 12. Demographics and Characteristics of Primary Patient Samples. (Related to FIG. 3) Patient demographics and clinical/cytomolecular characteristics of representative PRAME+/HLA-A2+ primary patient samples.

FIGS. 13A-13C. PRAME mTCRCAR T cells eliminate Pr20-positive patient-derived xenograft (PDX) AML cells in vivo. (FIG. 13A) Bioluminescent imaging of PDX leukemia-bearing mice treated with unmodified or PRAME mTCRCAR T cells, 5×10⁶ T cells per mouse. Shown are representative images at the indicated time points. (FIG. 13B) The Kaplan-Meier leukemia-free survival curves of PDX-leukemia-bearing mice untreated vs. treated with unmodified or PRAME mTCRCAR T cells. Statistical differences in survival were evaluated using log-rank Mantel-Cox tests. (FIG. 13C) Human leukemia cells detected in the peripheral blood is shown at representative time points from untreated vs. unmodified T cell treated vs. PRAME mTCRCAR T cell treated mice

FIG. 14. PRAME mTCRCAR T cells demonstrate in vitro efficacy against PRAME+/HLA-A2+ neuroblastoma cells. Cytolytic activity of CD8 T cells unmodified or transduced with PRAME mTCRCAR construct after 6 hours coculture with SK-N-SH cells. Data presented are mean specific lysis±SD from 3 technical replicates at indicated E:T ratios.

FIG. 15. Sequences supporting the disclosure.

### DETAILED DESCRIPTION

Adoptive transfer of T cells engineered to express chimeric antigen receptors (CAR) has achieved impressive outcomes in the treatment of refractory/relapsed B-cell acute lymphoblastic leukemia (B-ALL), providing potentially curative options for these patients. CAR generally include an extracellular component including a binding domain linked to an intracellular domain through a transmembrane domain. When the binding domain binds a marker, the intracellular component signals the immune cell to destroy the bound cell. The intracellular components provide such activation signals based on the inclusion of an effector domain. First generation CAR utilized the cytoplasmic domain of CD3ζ as an effector domain. Second generation CAR utilized the cytoplasmic domain of CD3ζ in combination with cluster of differentiation 28 (CD28) or 4-1BB (CD137) cytoplasmic domains, while third generation CAR have utilized the CD3ζ cytoplasmic domain in combination with the CD28 and 4-1BB cytoplasmic domains as effector domains.

Other subcomponents that can increase a CAR's function can also be used. For example, spacer regions can provide a CAR with additional conformational flexibility, often increasing the binding domain's ability to bind the targeted cell marker. The appropriate length of a spacer region within a particular CAR can depend on numerous factors including how close or far a targeted marker is located from the surface of an unwanted cell's membrane.

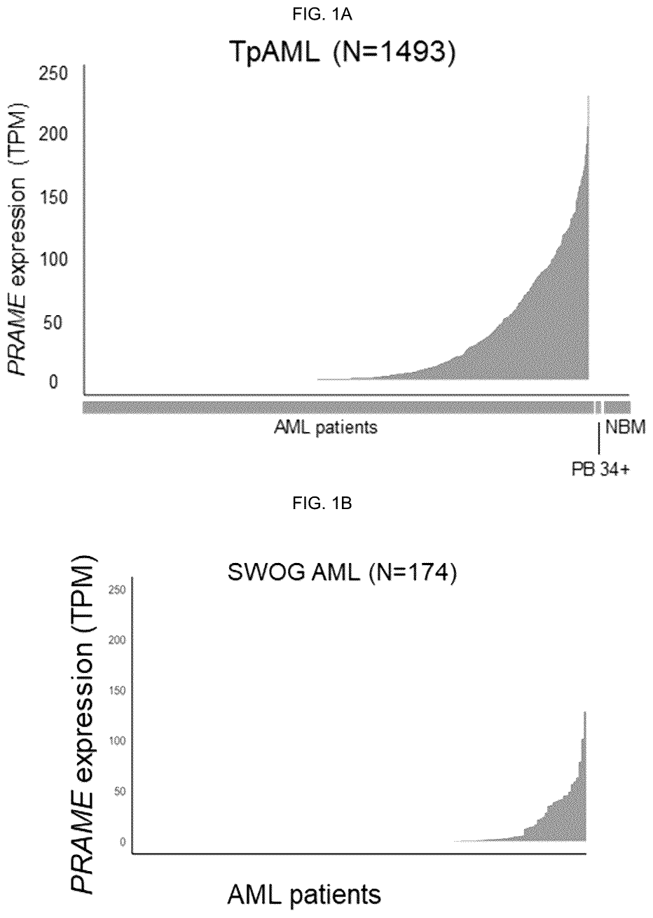

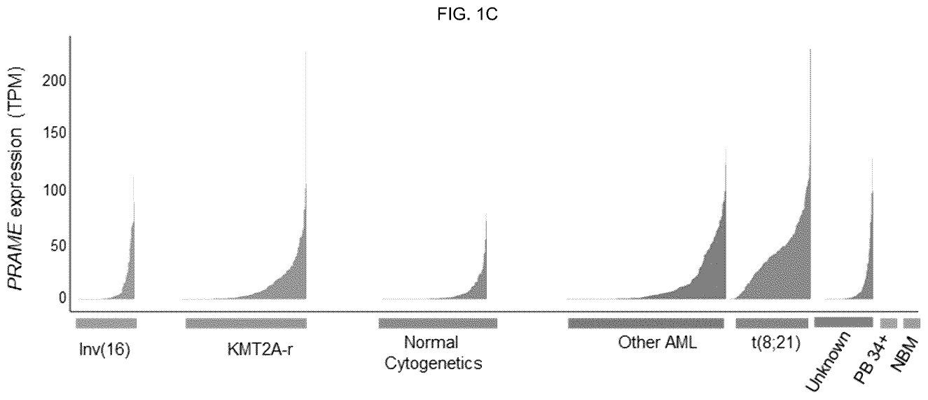

In an effort to identify AML-specific targets, the transcriptome of over 2000 AML cases in children and young adults was profiled and compared to normal hematopoiesis. Preferentially Expressed Antigen in Melanoma (PRAME) was identified as one of the highest expressing genes in AML that was not expressed in peripheral blood CD34+ and bone marrow samples (FIG. 1A), providing a promising target for immunotherapeutic development against AML.

PRAME is a cancer-testis antigen as its expression is restricted to the testes, ovaries, and endometrium in normal adult tissues (Al-Khadairi et al., 2019, 11(7):984; Chang et al., J Clin Invest. 2017, 127(9):3557; and Xu et al., Cell Prolif. 2020, 53(3):e12770). PRAME is overexpressed in a variety of cancers, including melanoma (Wadelin et al., Mol Cancer. 2010, 9:226; and Ikeda et al., Immunity. 1997, 6(2):199-208), neuroblastoma (Epping et al., Cancer Res. 2006, 6(22):10639-10642; and Oberthuer et al., Clin Cancer Res. 2004, 10(13):4307-4313), breast (Epping et al., Cancer Res. 2006, 6(22):10639-10642), ovarian (Tajeddine et al., Cancer Res. 2005, 65(16):7348-7355), cervical (Xu et al., Cell Prolif. 2020, 53(3):e12770), and lung cancers (Thongprasert et al., Lung Cancer. 2016, 101:137-144; Bankovic et al., Lung Cancer. 2010, 67(2):151-159; and Pan et al., Asia Pac J Clin Oncol. 2017, 13(5):e212-e223) as well as hematologic malignancies (Greiner et al., Int J Cancer. 2004, 108(5):704-711; Ding et al., Cancer Biol Med. 2012, 9(1):73-76; and Radich et al., Proc Natl Acad Sci USA. 2006, 103(8):2794-2799). PRAME binds to retinoic acid receptor and blocks retinoic acid-mediated proliferation arrest, differentiation, and apoptosis (Epping et al., Cell. 2005, 122(6):835-847). Depending on the specific cancer type, PRAME has been shown to act as both an oncogene or a tumor suppressor gene (Xu et al., Cell Prolif. 2020, 53(3):e12770). In breast cancer (Doolan et al., Breast Cancer Res Treat. 2008, 109(2):359-365), sarcoma (Xu et al., Cell Prolif. 2020, 53(3):e12770), and neuroblastoma (Oberthuer et al., Clin Cancer Res. 2004, 10(13):4307-4313), PRAME expression is associated with poor prognosis and increased risk for metastasis. In hematologic malignancies, PRAME expression has been shown to inhibit cell differentiation, growth arrest and apoptosis, while in other systems it can promote cell death, reduce tumorigenicity and increase sensitivity to chemotherapy (Xu et al., Cell Prolif. 2020, 53(3):e12770; Tajeddine et al., Cancer Res. 2005, 65(16):7348-7355; and Epping et al., Cell. 2005, 122(6):835-847).

Given its broad expression in cancer, PRAME is a promising target for immunotherapy. Since PRAME is an intracellular protein, it cannot be targeted by conventional CAR T cells which are restricted to cell surface antigens. Chang et al (J Clin Invest. 2017, 127(9):3557) developed a T cell receptor (TCR) mimic antibody called Pr20 that recognizes the peptide-HLA complex formed by PRAME ALY peptide and HLA-A2. Here, it is demonstrated that the Pr20 antigen is expressed on the cell surface in cell lines and primary AML blasts restricted to cells with HLA-A2 expression. Utilizing the Pr20 monoclonal antibody, CAR T cells targeting the PRAME antigen in AML (referred to as PRAME ^{mTCR}CAR T cells) were developed. PRAME ^{mTCR}CAR T cells demonstrate in vitro and in vivo efficacy against HLA-A2 restricted AML cells expressing the PRAME antigen, providing a novel approach to target PRAME with CAR T cells.

In particular embodiments, disclosed CAR include a binding domain that binds PRAME ALYVDSLFFL (SEQ ID NO: 94) (ALY)/HLA-A*0201 (HLA-A2). In particular embodiments, the binding domain that binds PRAME ALY(SEQ ID NO:94)/HLA-A2 is derived from the Pr20 antibody sequence.

In particular embodiments, the current disclosure provides CAR having an intermediate spacer region. In particular embodiments, the intermediate spacer region includes the hinge region and the CH3 domain of IgG4 (collectively, 131 amino acids). In particular embodiments, the spacer is a short spacer. In particular embodiments, the spacer is a long spacer.

In particular embodiments the current disclosure provides CAR having a transmembrane domain including the CD28 transmembrane domain. In particular embodiments, the current disclosure provides CAR having an intracellular effector domain including the 4-1BB and CD3ζ signaling domains.

The current disclosure also provides targeted therapeutics for the treatment of AML based on antibody formats, such as antibody-drug conjugates, antibody-radioisotope conjugates, antibody-immunotoxin conjugates, or antibody-nanoparticle conjugates.

In various embodiments, administration of the disclosed PRAME ^{mTCR}CAR T cells to a patient may increase the lysis of acute myeloid leukemia cells. In some aspects, the effectiveness of PRAME ^{mTCR}CAR T cells may be evaluated using in vitro studies. For example, the cytotoxicity of disclosed PRAME ^{mTCR}CAR T cells against AML2 cells includes a range of 55-85% cytotoxcity, 50-80% cytotoxcity, 45-75% cytotoxcity, or 40-70% cytotoxcity. In particular embodiments, the cytotoxicity of disclosed PRAME ^{mTCR}CAR T cells against OCI-AML2 cells includes a range of 60-70% cytotoxcity, 58-68% cytotoxcity, 52-62% cytotoxcity, or 50-60% cytotoxcity. In various embodiments, the cytotoxicity of disclosed PRAME ^{mTCR}CAR T cells against THP-1 cells includes a range of 55-85% cytotoxcity, 65-95% cytotoxcity, 55-85% cytotoxcity, or 35-65% cytotoxcity.

In particular embodiments, administration of the disclosed PRAME ^{mTCR}CAR T cells to a patient may increase lysis of AML cells by 10%-100%, 10%-75%, 10%-60%, 20%-90%, 30%-90%, 40%-90%, or 50%-90% over patients who are not treated with PRAME ^{mTCR}CAR. In some aspects, the effectiveness of PRAME ^{mTCR}CAR T cells may be evaluated using in vitro studies. For example, the PRAME ^{mTCR}CAR T cells described herein may increase the percent lysis of OCI-AML2 cells from a range of 10-60%, 40-90%, or 60-100% at 6 hours of co-incubation with disclosed PRAME ^{mTCR}CAR T cells, 12 hours of co-incubation with disclosed PRAME ^{mTCR}CAR T cells, and 24 hours of co-incubation with disclosed PRAME ^{mTCR}CAR T cells, respectively. In particular embodiments, the percent increase in percent lysis of AML2 cells includes a range of 20-40%, 20-60%, and 60-100%.

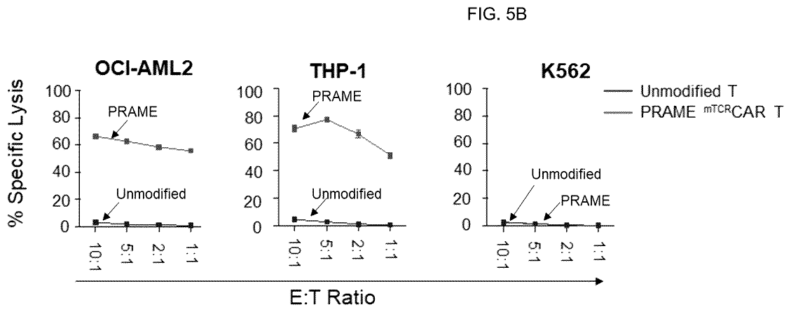

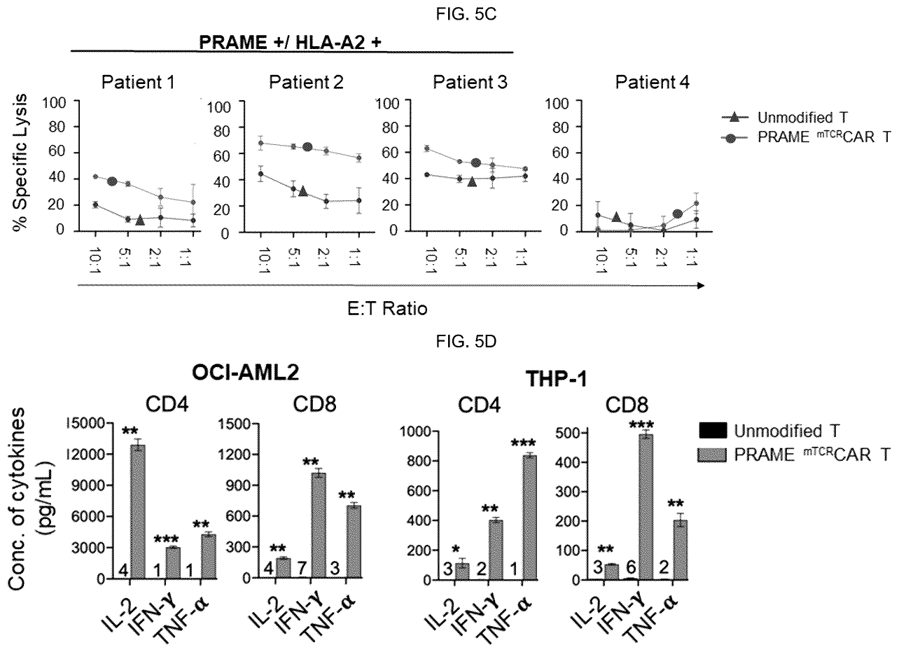

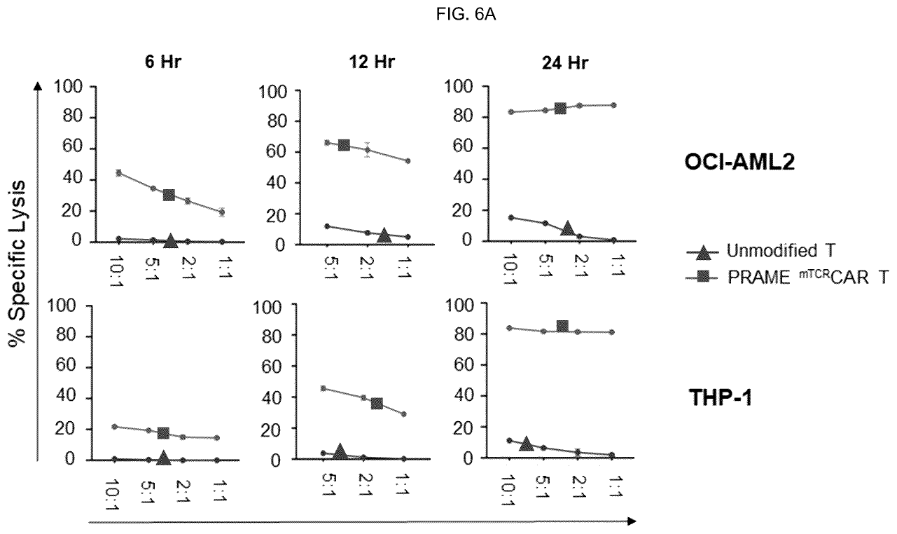

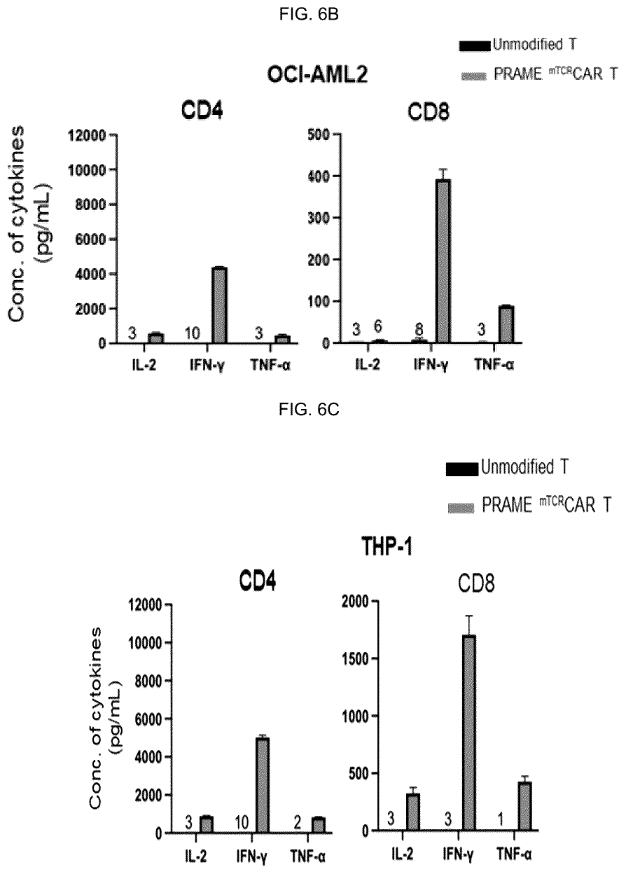

In some aspects administration of PRAME ^{mTCR}CAR T cells to a patient may increase cytokine production. The increase in cytokine production may be identified according to any method generally used by those of ordinary skill in the art. In some aspects, the increase in cytokine production may be relative to a reference level. Such a reference level may be an amount in a subject with AML who has not received PRAME ^{mTCR}CAR T cells, an amount in a healthy subject, and/or an amount in a subject prior to administration of PRAME ^{mTCR}CAR T cells. In particular embodiments, administration of PRAME ^{mTCR}CAR T cells may increase cytokine production by 10 fold, 20 fold, 50 fold, 60 fold, 70 fold, 80 fold, 100 fold, 130 fold, 140 fold, 800 fold, 1000 fold, 2000 fold, 3000 fold, 3250 fold, 4000 fold. Exemplary cytokines include IL-2, IFNγ, and TNFα. In some aspects administration of PRAME ^{mTCR}CAR T cells may increase cytokine production by CD4 cells, CD8 cells, or both. In some aspects, the increase in cytokine production of particular PRAME ^{mTCR}CAR T cells may be assessed in vitro as described herein and shown in FIGS. 5D, 6B, and 6C.

In various embodiments, PRAME ^{mTCR}CAR T cells may be co-administered with one or more cytokines. For example PRAME ^{mTCR}CAR T cells may be co-administered with IFNγ.

In various embodiments, administration of PRAME ^{mTCR}CAR T cells may decrease the number of live AML cells in a patient. In particular embodiments, administration of PRAME ^{mTCR}CAR T cells may decrease the number of live AML cells in a patient by 5%, 10%, 15%, or 20%. In some aspects, administration of PRAME ^{mTCR}CAR T cells may cause remission in a subject.

Particular embodiments utilize CAR disclosed herein to treat t(8;21) AML, Inv(16) AML, or KMT2A-r AML. Particular embodiments screen subjects for the presence of t(8;21) AML, Inv(16) AML, or KMT2A-r AML before initiating a treatment based on the screening. Particular embodiments include co-administering interferon gamma (IFNγ) to increase PRAME expression by cancer cells.

Aspects of the current disclosure are now described with additional detail and options as follows: (i) Immune Cells; (ii) Cell Sample Collection and Cell Enrichment; (iii) Genetically Modifying Cell Populations to Express Chimeric Antigen Receptors (CAR); (iii-a) Genetic Engineering Techniques; (iii-b) CAR Subcomponents; (iii-b-i) Binding Domains; (iii-b-ii) Spacer Regions; (iii-b-iii) Transmembrane Domains; (iii-b-iv) Intracellular Effector Domains; (iii-b-v) Linkers; (iii-b-vi) Control Features Including Tag Cassettes, Transduction Markers, and/or Suicide Switches; (iii-b-vii) Multimerization Domains; (iv) Cell Activating Culture Conditions; (v) Ex Vivo Manufactured Cell Formulations; (vi) Compositions; (vii) Methods of Use; (viii) Exemplary Embodiments; (ix) Experimental Example; and (x) Closing Paragraphs. These headings are provided for organizational purposes only and do not limit the scope or interpretation of the disclosure.

### (i) IMMUNE CELLS

The present disclosure describes cells genetically modified to express CAR. Genetically modified cells can include T-cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC). In particular embodiments, genetically modified cells include T-cells.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γ⁶ T-cells, the TCR is made up of one γ-chain and one δ-chain. This group of T-cells is much less common (2% of total T-cells) than the αβ T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) as used herein refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+T lymphocytes are characterized by the expression of phenotypic markers of naive T-cells including CD62L, CCR7, CD28, CD127, and CD45RA.

Natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

NK cells include NK-T cells. NK-T cells are a specialized population of T cells that express a semi invariant T cell receptor (TCR ab) and surface antigens typically associated with natural killer cells. NK-T cells contribute to antibacterial and antiviral immune responses and promote tumor-related immunosurveillance or immunosuppression. Like natural killer cells, NK-T cells can also induce perforin-, Fas-, and TNF-related cytotoxicity. Activated NK-T cells are capable of producing IFN-γ and IL-4. In particular embodiments, NK-T cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Rα; and/or CD163.

Immature dendritic cells (i.e., pre-activation) engulf antigens and other non-self-components in the periphery and subsequently, in activated form, migrate to T-cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD24^{lo} Lin⁻ CD117+ hematopoietic progenitor cells. HPC can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. Preferably, the HSPC are CD34⁺ cells.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker. In particular embodiments, a surface marker and surface expression includes the presentation of a PRAME peptide or fragment thereof on a major histocompatibility complex (MHC) molecule.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

In particular embodiments, immune cells to be genetically modified according to the teachings of the current disclosure can be patient-derived cells (autologous) or allogeneic when appropriate and can also be in vivo or ex vivo. In particular embodiments, cells to be genetically modified include CD4+ or CD8+ T cells.

### (ii) CELL SAMPLE COLLECTION AND CELL ENRICHMENT

Methods of sample collection and enrichment are known by those skilled in the art. In some embodiments, cells are derived from cell lines. In particular embodiments, cells are derived from humans for example a patient to be treated. Cells can be derived from cell lines. The cells in some embodiments are obtained from a xenogeneic source, for example, from mouse, rat, non-human primate, or pig.

In some embodiments, T cells are derived or isolated from samples such as whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T cells, monocytes, granulocytes, B cells, other nucleated white blood cells, HSC, HPC, HSPC, red blood cells, and/or platelets, and in some aspects contains cells other than red blood cells and platelets and further processing is necessary.

In some embodiments, blood cells collected from a subject are washed, e.g., to remove the plasma fraction and to place the cells in an appropriate buffer or media for subsequent processing steps.

Isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or engineering of the different populations is carried out from the same starting material, such as from the same sample.

In particular embodiments, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, a selected T cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some embodiments, an antibody or binding domain for a cellular marker is bound to a solid support or matrix, such as a magnetic bead or paramagnetic bead, to allow for separation of cells for positive and/or negative selection.

In some embodiments, affinity-based selection is via magnetic-activated cell sorting (MACS) (Miltenyi Biotec, Auburn, CA). MACS systems are capable of high-purity selection of cells having magnetized particles attached thereto.

In some embodiments, a cell population described herein is collected and enriched (or depleted) via flow cytometry, in which cells stained for multiple cell surface markers are carried in a fluidic stream.

Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques.

CD3+, CD28+ T cells can be positively selected for and expanded using anti-CD3/anti-CD28 conjugated magnetic beads (e.g., DYNABEADS® M-450 CD3/CD28 T Cell Expander).

In particular embodiments, a CD8+ or CD4+ selection step is used to separate CD4+ helper and CD8+ cytotoxic T cells. Such CD8+ and CD4+ populations can be further sorted into sub-populations by positive or negative selection for markers expressed or expressed to a relatively higher degree on one or more naive, memory, and/or effector T cell subpopulations.

In some embodiments, enrichment for central memory T (TCM) cells is carried out. In particular embodiments, memory T cells are present in both CD62L subsets of CD8+ peripheral blood lymphocytes. PBMC can be enriched for or depleted of CD62L, CD8 and/or CD62L+CD8+ fractions, such as by using anti-CD8 and anti-CD62L antibodies.

In some embodiments, the enrichment for central memory T (TCM) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CCR7, CD45RO, and/or CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L.

Other cell types can be enriched based on known marker profiles and techniques. For example, CD34+ HSC, HSP, and HSPC can be enriched using anti-CD34 antibodies directly or indirectly conjugated to magnetic particles in connection with a magnetic cell separator, for example, the CliniMACS® Cell Separation System (Miltenyi Biotec, Bergisch Gladbach, Germany).

### (iii) GENETICALLY MODIFYING CELL POPULATIONS TO EXPRESS CHIMERIC ANTIGEN RECEPTORS (CAR)

Cell populations are genetically modified to express chimeric antigen receptors (CAR) described herein.

(iii-a) Genetic Engineering Techniques. Desired genes encoding CAR disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence) that encodes a CAR. This definition includes various sequence polymorphisms, mutations, and/or sequence variants wherein such alterations do not substantially affect the function of the encoded CAR. The term “gene” may include not only coding sequences but also regulatory regions such as promoters, enhancers, and termination regions. Gene sequences encoding the molecule can be DNA or RNA that directs the expression of the CAR. The sequences can also include degenerate codons of the native sequence or sequences that may be introduced to provide codon preference in a specific cell type. Portions of complete gene sequences are referenced throughout the disclosure as is understood by one of ordinary skill in the art.

Gene sequences encoding CAR (also referred to as genetic constructs) are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In embodiments, the gene sequence encoding the sequences can be codon optimized for expression in mammalian cells.

“Encoding” refers to the property of specific sequences of nucleotides in a gene, such as a cDNA, or an mRNA, to serve as templates for synthesis of other macromolecules such as a defined sequence of amino acids.

Polynucleotide gene sequences encoding more than one portion of an expressed CAR can be operably linked to each other and relevant regulatory sequences. For example, there can be a functional linkage between a regulatory sequence and an exogenous nucleic acid sequence resulting in expression of the latter. For instance, a promoter is operably linked to a coding sequence if the promoter affects the transcription or expression of the coding sequence.

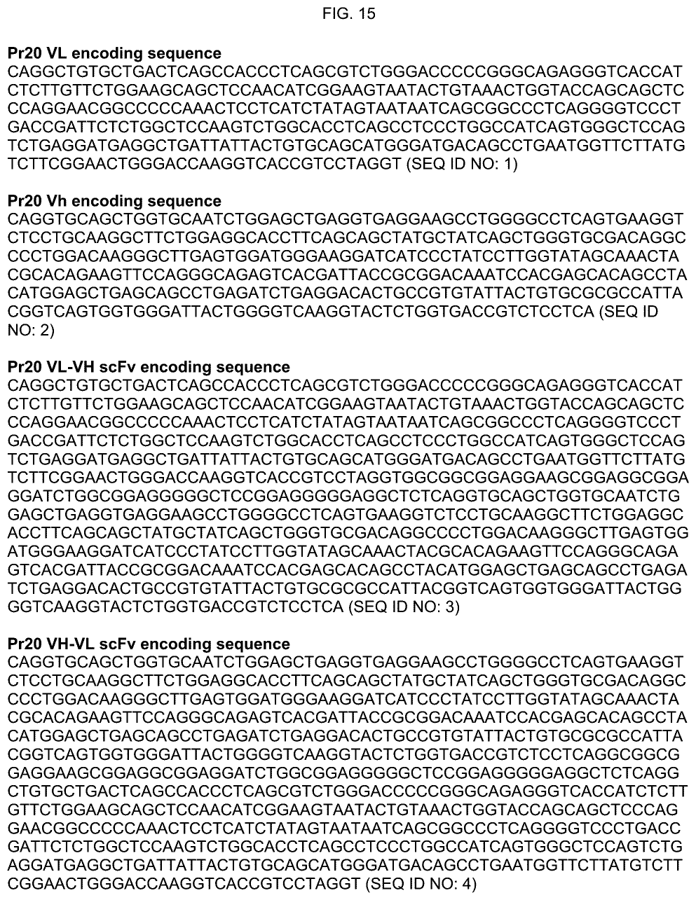

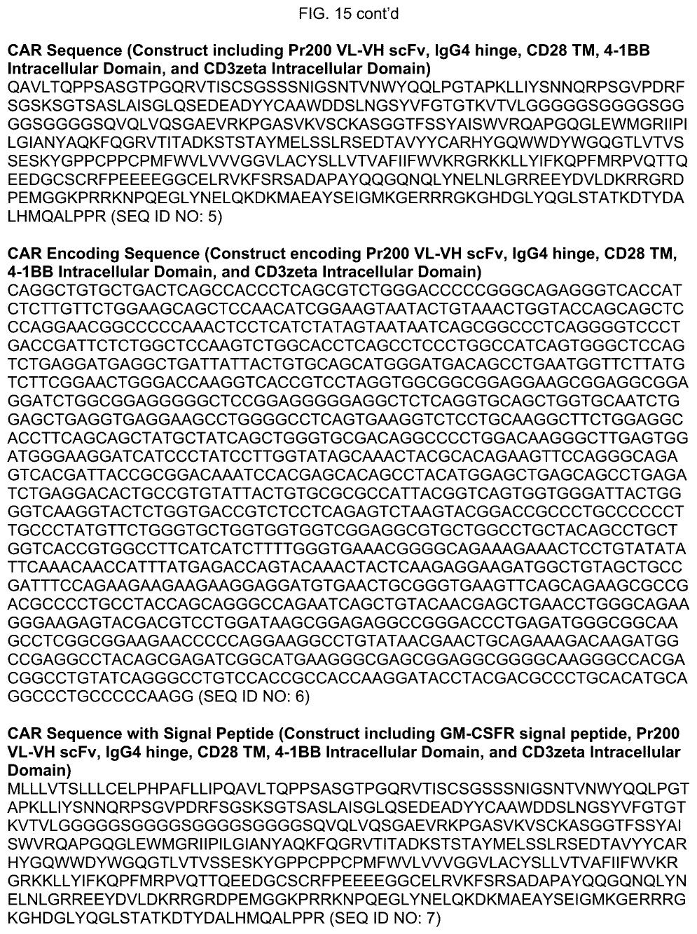

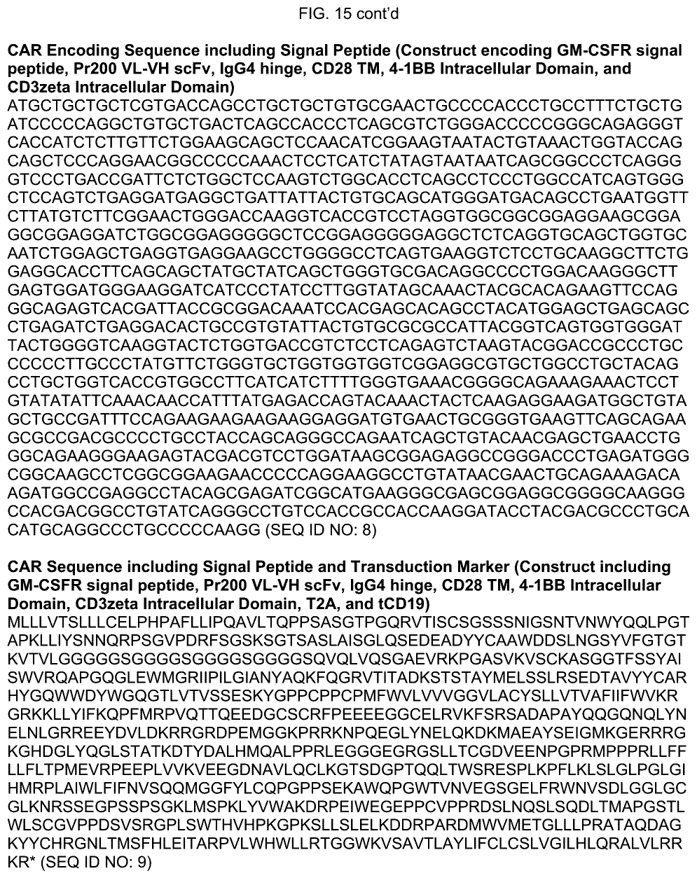

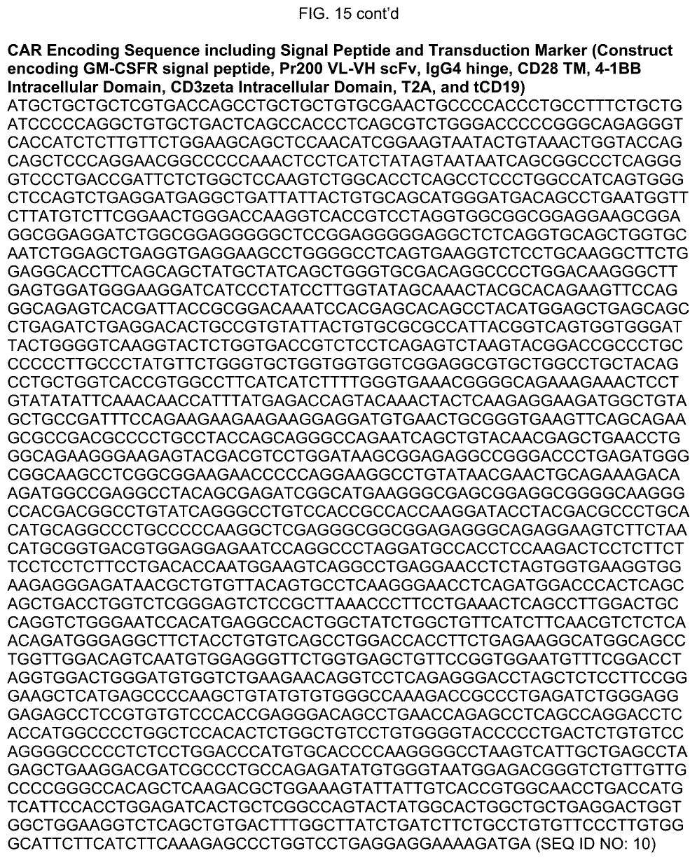

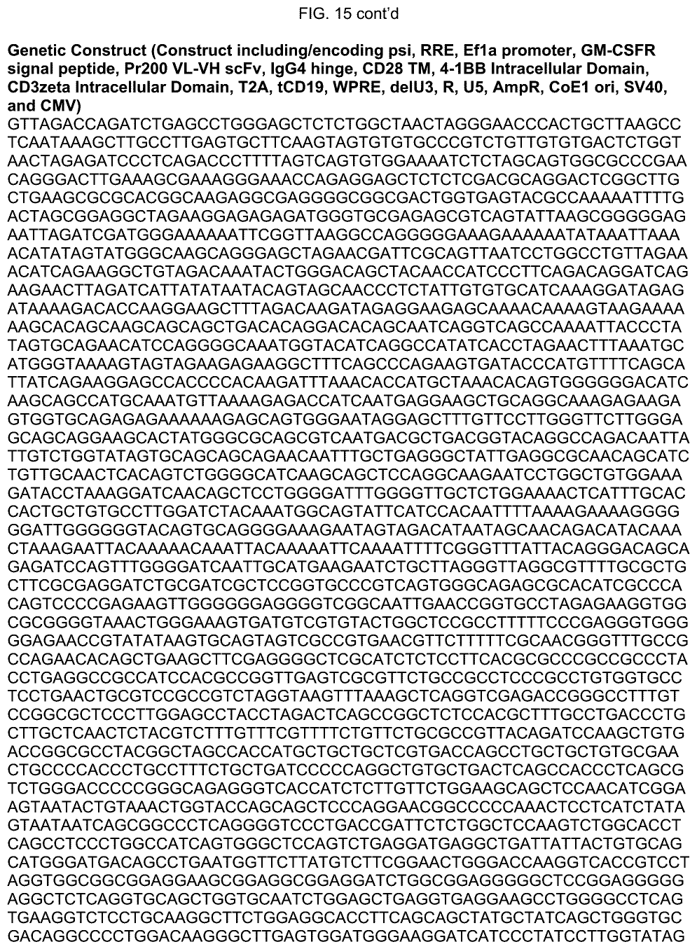

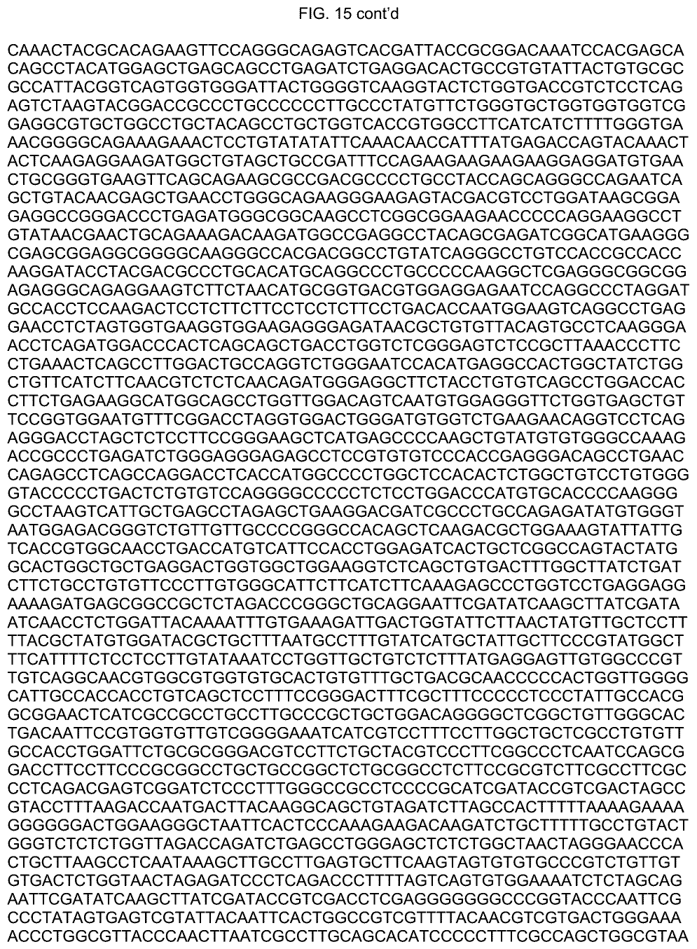

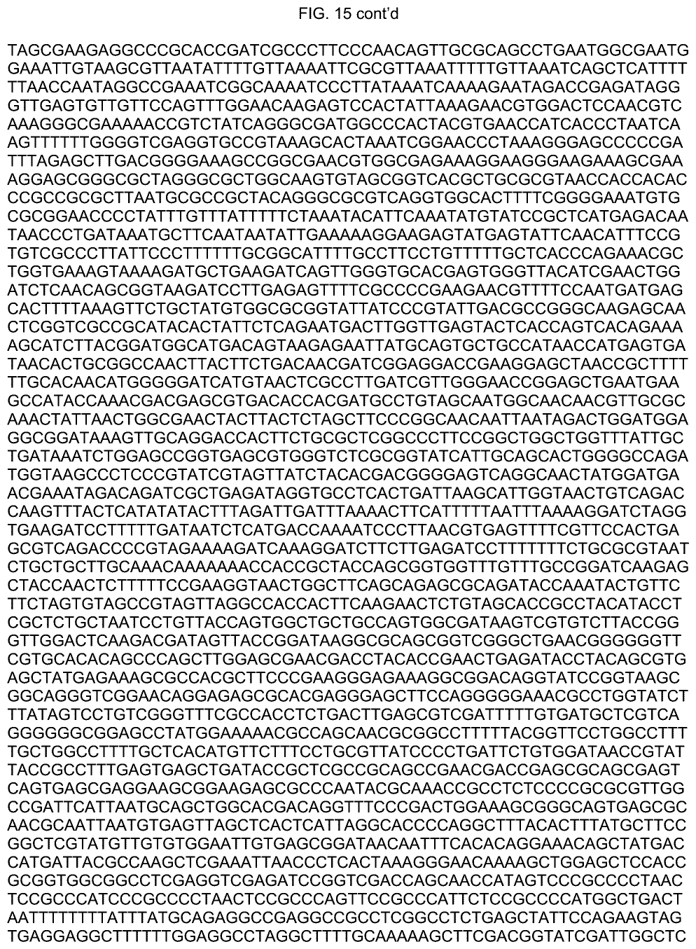

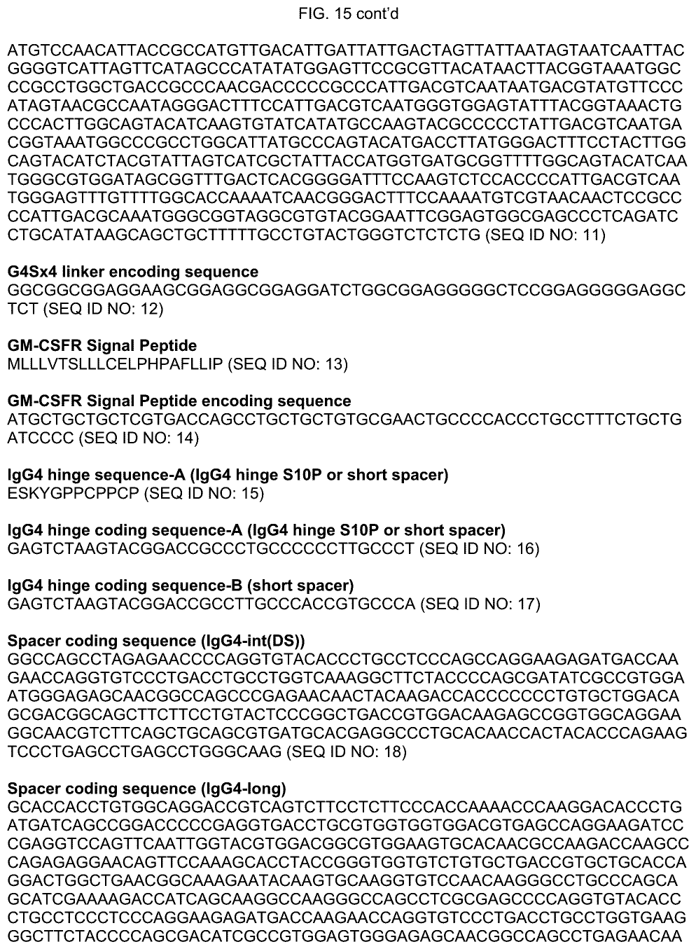

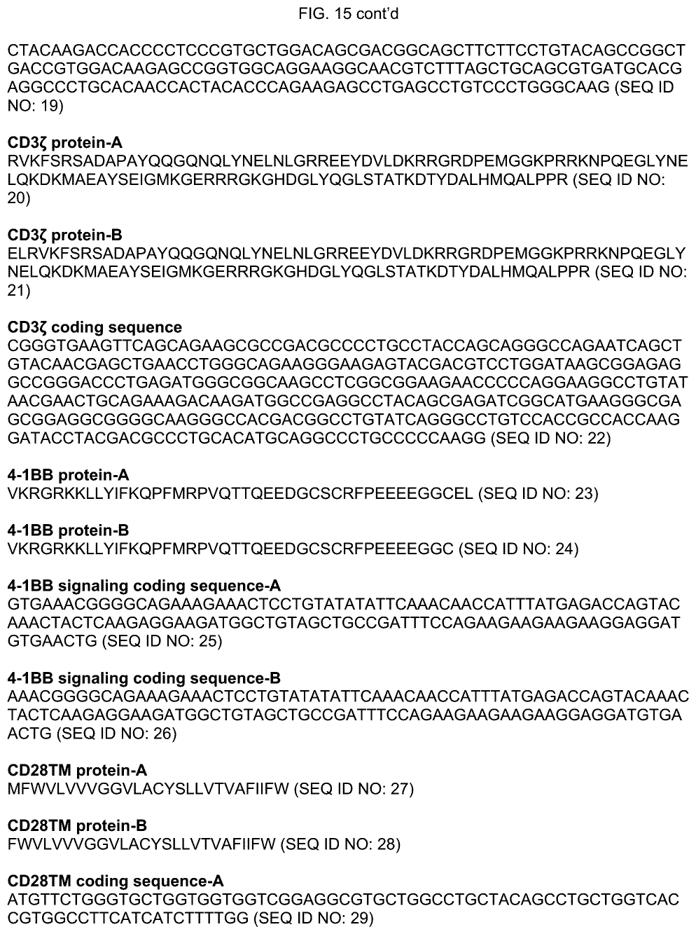

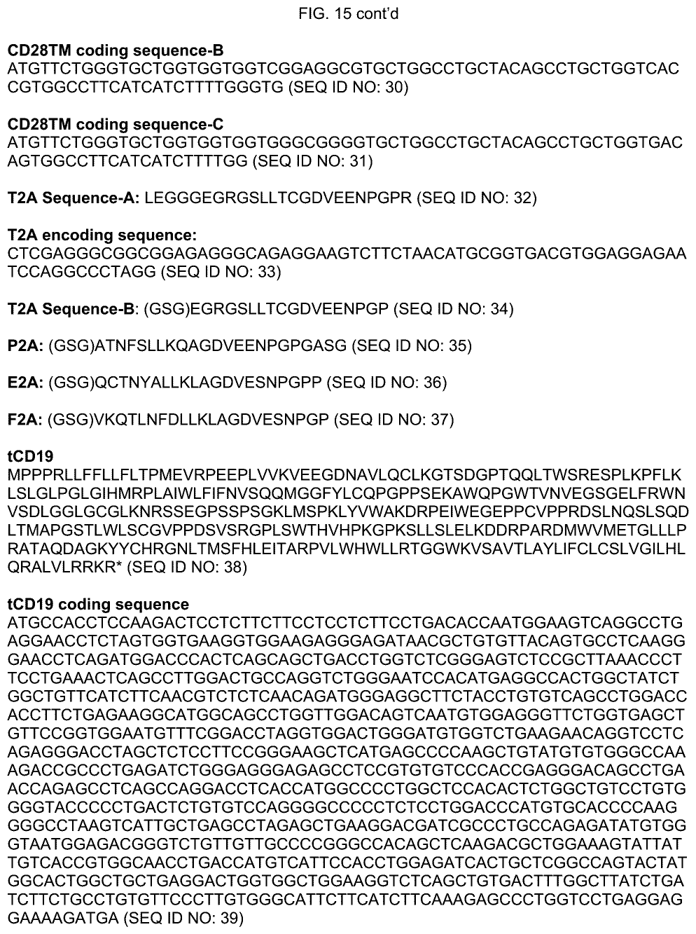

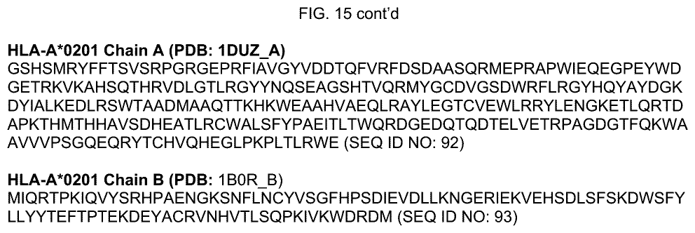

In any of the embodiments described herein, a polynucleotide can include a polynucleotide that encodes a self-cleaving polypeptide, wherein the polynucleotide encoding the self-cleaving polypeptide is located between the polynucleotide encoding the CAR construct and a polynucleotide encoding a transduction marker (e.g., tCD19 or tEGFR). Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof (see FIG. 15). Further exemplary nucleic acid and amino acid sequences of 2A peptides are set forth in, for example, Kim et al. (PLOS One 6:e18556 (2011).

A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et ah, Mol. Ther. 17(8): 1453-1464 (2009). Other examples of lentivirus vectors that may be used in the clinic, include: the LENTIVECTOR® gene delivery technology from Oxford BioMedica, the LENTIMAX™ vector system from Lentigen and the like.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

Retroviral vectors (see Miller, et al., 1993, Meth. Enzymol. 217:581-599) can be used. In such embodiments, the gene to be expressed is cloned into the retroviral vector for its delivery into cells. In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses, adeno-associated viruses (AAV) and alphaviruses can also be used. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686. Other methods of gene delivery include use of mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including CAR transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. For information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. For information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Nanoparticles that result in selective in vivo genetic modification of targeted cell types can be used within the teachings of the current disclosure. In particular embodiments, the nanoparticles can be those described in WO2014153114, WO2017181110, and WO201822672.

In particular embodiments, T cells are transduced with a lentivirus encoding CAR.

(iii-b) CAR Subcomponents. As described previously, CAR molecules include several distinct subcomponents that allow genetically modified cells to recognize and kill unwanted cells, such as cancer cells. The subcomponents include at least an extracellular component and an intracellular component. The extracellular component includes a binding domain that specifically binds a marker that is preferentially present on the surface of unwanted cells. When the binding domain binds such markers, the intracellular component activates the cell to destroy the bound cell. CAR additionally include a transmembrane domain that links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted cell marker.

(iii-b-i) Binding Domains. The current disclosure provides CAR with a binding domain binds PRAME SEQ ID NO: 94 ALYVDSLFFL (ALY)/HLA-A*0201 (HLA-A2) (PRAME ALY/HLA-A2).

Particular embodiments include binding domains derived from antibodies. Antibodies include whole antibodies or binding fragments of an antibody, e.g., Fv, Fab, Fab′, F(ab′)2, and single chain (sc) forms and fragments thereof that specifically bind a cellular marker (such as PRAME ALY/HLA-A2). Antibodies or antigen binding fragments can include all or a portion of polyclonal antibodies, monoclonal antibodies, human antibodies, humanized antibodies, synthetic antibodies, non-human antibodies, recombinant antibodies, chimeric antibodies, bispecific antibodies, mini bodies, and linear antibodies.

Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR).

The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al. (1997) J Mol Biol 273: 927-948 (Chothia numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); North et al. (2011) J. Mol. Biol. 406(2):228-56 (North numbering scheme); Lefranc M P et al. (2003) Dev Comp Immunol 27(1): 55-77 (IMGT numbering scheme); and Honegger and Pluckthun (2001) J Mol Biol 309(3): 657-670 (“Aho” numbering scheme). CDR residues can be identified using software programs such as ABodyBuilder. The boundaries of a given CDR or FR may vary depending on the scheme used for identification.

In particular embodiments, the amino acid sequence for human PRAME (GenBank: CAG30435.1) includes the sequence:

(SEQ ID NO: 40)
MERRRLRGSIQSRYISMSVWTSPRRLVELAGQSLLKDEALAIAALELLPR
ELFPPLFMAAFDGRHSQTLKAMVQAWPFTCLPLGVLMKGQHLHLETFKAV
LDGLDVLLAQEVRPRRWKLQVLDLRKNSHQDFWTVWSGNRASLYSFPEPE
AAQPMTKKRKVDGLSTEAEQPFIPVEVLVDLFLKEGACDELFSYLIEKVK
RKKNVLRLCCKKLKIFAMPMQDIKMILKMVQLDSIEDLEVTCTWKLPTLA
KFSPYLGQMINLRRLLLSHIHASSYISPEKEEQYIAQFTSQFLSLQCLQA
LYVDSLFFLRGRLDQLLRHVMNPLETLSITNCRLSEGDVMHLSQSPSVSQ
LSVLSLSGVMLTDVSPEPLQALLERASATLQDLVFDECGITDDQLLALLP
SLSHCSQLTTLSFYGNSISISALQSLLQHLIGLSNLTHVLYPVPLESYED
IHGTLHLERLAYLHARLRELLCELGRPSMVWLSANPCPHCGDRTFYDPEP
ILCPCFMPN.

In particular embodiments, a binding domain that binds PRAME ALY/HLA-A2 includes an scFv derived from the Pr20 antibody. In particular embodiments, the variable light chain of Pr20 includes the sequence:

(SEQ ID NO: 41)
QAVLTQPPSASGTPGQRVTISCSGSSSNIGSNTVNWYQQLPGTAPKLLIY
SNNQRPSGVPDRFSGSKSGTSASLAISGLQSEDEADYYCAAWDDSLNGSY
VFGTGTKVTVLG.

In particular embodiments, the variable heavy chain of the Pr20 antibody includes the sequence:

(SEQ ID NO: 42)
QVQLVQSGAEVRKPGASVKVSCKASGGTFSSYAISWVRQAPGQGLEWMGR
IIPILGIANYAQKFQGRVTITADKSTSTAYMELSSLRSEDTAVYYCARHY
GQWWDYWGQGTLVTVSS.

In particular embodiments, an exemplary scFv derived from the Pr20 antibody includes the sequence:

(SEQ ID NO: 43)
QAVLTQPPSASGTPGQRVTISCSGSSSNIGSNTVNWYQQLPGTAPKLLIY
SNNQRPSGVPDRFSGSKSGTSASLAISGLQSEDEADYYCAAWDDSLNGSY
VFGTGTKVTVLGGGGGSGGGGSGGGGSGGGGSQVQLVQSGAEVRKPGASV
KVSCKASGGTFSSYAISWVRQAPGQGLEWMGRIIPILGIANYAQKFQGRV
TITADKSTSTAYMELSSLRSEDTAVYYCARHYGQWWDYWGQGTLVTVSS.

In particular embodiments, an exemplary scFv derived from the Pr20 antibody includes the sequence:

(SEQ ID NO: 44)
QVQLVQSGAEVRKPGASVKVSCKASGGTFSSYAISWVRQAPGQGLEWMGR
IIPILGIANYAQKFQGRVTITADKSTSTAYMELSSLRSEDTAVYYCARHY
GQWWDYWGQGTLVTVSSGGGGSGGGGSGGGGSGGGGSQAVLTQPPSASGT
PGQRVTISCSGSSSNIGSNTVNWYQQLPGTAPKLLIYSNNQRPSGVPDRF
SGSKSGTSASLAISGLQSEDEADYYCAAWDDSLNGSYVFGTGTKVTVLG.

In some instances, additional scFvs based on the binding domains described herein and for use in a CAR can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies.

Referring to PRAME ALY/HLA-A2 binding domains provided herein, the following CDR sets can be used to generate a PRAME ALY/HLA-A2 binding domain of a CAR disclosed herein. A CDR set refers to 3 light chain CDRs and 3 heavy chain CDRs that together result in binding to PRAME ALY/HLA-A2.

TABLE 1
PRAME ALY/HLA-A2 CDR Binding Set Sequences.

| CDR | | | SEQ ID |
|---|---|---|---|
| Definition | CDR | Sequence | NO: |
| North | CDRH1 | KASGGTFSSYAIS | 45 |
| | CDRH2 | RIIPILGIAN | 46 |
| | CDRH3 | ARHYGQWWDY | 47 |
| | CDRL1 | SGSSSNIGSNTVN | 48 |
| | CDRL2 | YSNNQRPS | 49 |
| | CDRL3 | AAWDDSLNGSYV | 50 |
| Kabat | CDRH1 | SYAIS | 51 |
| | CDRH2 | RIIPILGIANYAQKFQG | 52 |
| | CDRH3 | HYGQWWDY | 53 |
| | CDRL1 | SGSSSNIGSNTVN | 48 |
| | CDRL2 | YSNNQRPS | 49 |
| | CDRL3 | AAWDDSLNGSYV | 50 |
| IMGT | CDRH1 | GGTFSSYA | 54 |
| | CDRH2 | IIPILGIA | 55 |
| | CDRH3 | ARHYGQWWDY | 47 |
| | CDRL1 | SSNIGSNT | 56 |
| | CDRL2 | SNN | N/A |
| | CDRL3 | AAWDDSLNGSYV | 50 |
| Chothia | CDRH1 | GGTFSSYA | 54 |
| | CDRH2 | IPILGI | 57 |
| | CDRH3 | HYGQWWDY | 53 |
| | CDRL1 | SGSSSNIGSNTVN | 48 |
| | CDRL2 | SNNQRPS | 58 |
| | CDRL3 | AAWDDSLNGSYV | 50 |
| Contact | CDRH1 | SSYAIS | 59 |
| | CDRH2 | WMGRIIPILGIAN | 60 |
| | CDRH3 | ARHYGQWWD | 61 |
| | CDRL1 | SNTVNWY | 62 |
| | CDRL2 | LLIYSNNQRP | 63 |
| | CDRL3 | AAWDDSLNGSY | 64 |

CDR predictions were generated using the program SAbPrep http://opig.stats.ox.ac.uk/webapps/newsabdab/sabpred/). ABodyBuilder within SAbPred was used (CDR predictions based on “Clothia”).

Although chimeric antibodies often incorporate all six CDRs from a non-human antibody, they can also be made with less than all CDRs (e.g., at least 3, 4, or 5) CDRs from a non-human antibody (e.g., Pascalis et al., J. Immunol. 169:3076, 2002; Vajdos et al., Journal of Molecular Biology, 320: 415-428, 2002; Iwahashi et al., Mol. Immunol.

Other binding fragments, such as Fv, Fab, Fab′, F(ab′)2, can also be used within the CAR disclosed herein.

In particular embodiments, a VL region in a binding domain includes one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the antibody disclosed herein. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region in a binding domain includes one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH disclosed herein. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from an antibody disclosed herein or fragment or derivative thereof that specifically binds PRAME ALY/HLA-A2.

(iii-b-ii) Spacer Regions. Spacer regions are used to create appropriate distances and/or flexibility from other CAR sub-components. In particular embodiments, the length of a spacer region is customized for binding targeted cells and mediating destruction. In particular embodiments, a spacer region length can be selected based upon the location of a cellular marker epitope, affinity of a binding domain for the epitope, and/or the ability of the targeting agent to mediate cell destruction following target binding.

Spacer regions typically include those having 10 to 250 amino acids, 10 to 200 amino acids, 10 to 150 amino acids, 10 to 100 amino acids, 10 to 50 amino acids, or 10 to 25 amino acids.

In particular embodiments, a spacer region is 5 amino acids, 8 amino acids, 10 amino acids, 12 amino acids, 14 amino acids, 20 amino acids, 21 amino acids, 26 amino acids, 27 amino acids, 45 amino acids, 50 amino acids, or 75 amino acids. These lengths qualify as short spacer regions.

In particular embodiments, a spacer region is 76 amino acids, 100 amino acids, 110 amino acids, 120 amino acids, 125 amino acids, 128 amino acids, 131 amino acids, 135 amino acids, 140 amino acids, 150 amino acids, 160 amino acids, or 179 amino acids. These lengths qualify as intermediate spacer regions.

In particular embodiments, a spacer region is 180 amino acids, 190 amino acids, 200 amino acids, 210 amino acids, 212 amino acids, 214 amino acids, 216 amino acids, 218 amino acids, 220 amino acids, 230 amino acids, 240 amino acids, or 250 amino acids. These lengths qualify as long spacer regions.

In particular embodiments, spacer regions include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular embodiments, the spacer is a short spacer including an IgG4 hinge region. In particular embodiments the short spacer is encoded by either of SEQ ID NO: 16 or SEQ ID NO: 17. In particular embodiments the short spacer includes the sequence as set forth in SEQ ID NO: 15. In particular embodiments, the spacer is an intermediate spacer including an IgG4 hinge region and an IgG4 hinge CH3 region. In particular embodiments the intermediate spacer is encoded by SEQ ID NO: 18. In particular embodiments, the spacer is a long spacer including an IgG4 hinge region, an IgG4 CH3 region, and an IgG4 CH2 region. In particular embodiments the long spacer is encoded by SEQ ID NO: 19.

Other examples of hinge regions that can be used in CAR described herein include the hinge region present in the extracellular regions of type 1 membrane proteins, such as CD8a, CD4, CD28 and CD7, which may be wild-type or variants thereof.

In particular embodiments, a spacer region includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

(iii-b-iii) Transmembrane Domains. As indicated, transmembrane domains within a CAR serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2RP, IL2Ry, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge. In particular embodiments, the CAR includes a CD28 transmembrane domain. It has been shown that a CD28 transmembrane domain reduces the antigen-threshold for second-generation 4-1BB CAR T cell activation.

In particular embodiments, a transmembrane domain has a three-dimensional structure that is thermodynamically stable in a cell membrane, and generally ranges in length from 15 to 30 amino acids. The structure of a transmembrane domain can include an a helix, a β barrel, a β sheet, a β helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is encoded by the nucleic acid sequence encoding the CD28 transmembrane domain (SEQ ID NO: 29, SEQ ID NO: 30 or SEQ ID NO: 31). In particular embodiments, the transmembrane domain includes the amino acid sequence of the CD28 transmembrane domain (SEQ ID NO: 27 or SEQ ID NO: 28).

(iii-b-iv) Intracellular Effector Domains. The intracellular effector domains of a CAR are responsible for activation of the cell in which the CAR is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8a, CD8P, IL2RP, IL2Ry, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46. In particular embodiments, the effector domain includes a CD3ζ signaling domain.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8a, CD8P, IL2RP, IL2Ry, IL7Rα, ITGA4, VLA1, CD49a, ITGA4, IA4, CD49D, ITGA6, VLA-6, CD49f, ITGAD, CDIId, ITGAE, CD103, ITGAL, CDIIa, ITGAM, CDI lb, ITGAX, CDIIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a. In particular embodiments, the co-stimulatory domain includes a 4-1BB signaling domain.

In particular embodiments, the nucleic acid sequences encoding the intracellular signaling components includes CD3ζ encoding sequence (SEQ ID NO: 22) and 4-1BB signaling encoding sequence (SEQ ID NO: 25 and SEQ ID NO: 26). In particular embodiments, the amino acid sequence of the intracellular signaling component includes a CD3ζ (SEQ ID NO: 20 and SEQ ID NO: 21) and a portion of the 4-1BB (SEQ ID NO: 23 or SEQ ID NO: 24) intracellular signaling component.

In particular embodiments, the intracellular signaling component includes (i) all or a portion of the signaling domain of CD3ζ, (ii) all or a portion of the signaling domain of 4-1BB, or (iii) all or a portion of the signaling domain of CD3ζ and 4-1 BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

(iii-b-v) Linkers. As used herein, a linker can include a chemical moiety that serves to connect two other subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of a CAR.

Linkers can be flexible, rigid, or semi-rigid, depending on the desired function of the linker. Linkers can include junction amino acids. For example, in particular embodiments, linkers provide flexibility and room for conformational movement between different components of CAR. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 65), (Gly₃Ser)ₙ(Gly₄Ser)ₙ (SEQ ID NO: 66), (Gly₃Ser)ₙ(Gly₂Ser)ₙ (SEQ ID NO: 67), or (Gly₃Ser)ₙ(Gly₄Ser)₁ (SEQ ID NO: 68). In particular embodiments, the linker is (Gly₄Ser)₄ (SEQ ID NO: 69), (Gly₄Ser)₃ (SEQ ID NO: 70), (Gly₄Ser)₂ (SEQ ID NO: 71), (Gly₄Ser)₁ (SEQ ID NO: 72), (Gly₃Ser)₂ (SEQ ID NO: 73), (Gly₃Ser)₁ (SEQ ID NO: 74), (Gly₂Ser)₂ (SEQ ID NO: 75) or (Gly₂Ser)₁, GGSGGGSGGSG (SEQ ID NO: 76), GGSGGGSGSG (SEQ ID NO: 77), or GGSGGGSG (SEQ ID NO: 78).

In particular embodiments, a linker region is (GGGGS)ₙ (SEQ ID NO: 65) wherein n is an integer including, 1, 2, 3, 4, 5, 6, 7, 8, 9, or more. In particular embodiments, the spacer region is (EAAAK)ₙ (SEQ ID NO: 79) wherein n is an integer including 1, 2, 3, 4, 5, 6, 7, 8, 9, or more.

In some situations, flexible linkers may be incapable of maintaining a distance or positioning of CAR needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer region is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

(iii-b-vi) Control Features Including Tag Cassettes, Transduction Markers, and/or Suicide Switches. In particular embodiments, CAR constructs can include one or more tag cassettes and/or transduction markers. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a CAR, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the CAR molecule.

In particular embodiments, CAR include a T2A ribosomal skip element that separates the expressed CAR from a truncated CD19 (tCD19) transduction marker.

Tag cassettes that bind cognate binding molecules include, for example, His tag (HHHHHH; SEQ ID NO: 80), Flag tag (DYKDDDDK; SEQ ID NO: 81), Xpress tag (DLYDDDDK; SEQ ID NO: 82), Avi tag (GLNDIFEAQKIEWHE; SEQ ID NO: 83), Calmodulin tag (KRRWKKNFIAVSAANRFKKISSSGAL; SEQ ID NO: 84), Polyglutamate tag, HA tag (YPYDVPDYA; SEQ ID NO: 85), Myctag (EQKLISEEDL; SEQ ID NO: 86), Strep tag (which refers the original STREP® tag (WRHPQFGG; SEQ ID NO: 87), STREP® tag II (WSHPQFEK SEQ ID NO: 88 (IBA Institut fur Bioanalytik, Germany); see, e.g., U.S. Pat. No. 7,981,632), Softag 1 (SLAELLNAGLGGS; SEQ ID NO: 89), Softag 3 (TQDPSRVG; SEQ ID NO: 90), and V5 tag (GKPIPNPLLGLDST; SEQ ID NO: 91).

Conjugate binding molecules that specifically bind tag cassette sequences disclosed herein are commercially available. For example, His tag antibodies are commercially available from suppliers including Life Technologies, Pierce Antibodies, and GenScript.Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers may be selected from at least one of a truncated CD19 (tCD19; see Budde et al., Blood 122: 1660, 2013); a truncated human EGFR (tEGFR; see Wang et al., Blood 118: 1255, 2011); an ECD of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278).

In particular embodiments, a polynucleotide encoding an iCaspase9 construct (iCasp9) may be inserted into a CAR construct as a suicide switch.

Control features may be present in multiple copies in a CAR or can be expressed as distinct molecules with the use of a skipping element (SEQ ID NOs: 32, or 34-37). For example, a CAR can have one, two, three, four or five tag cassettes and/or one, two, three, four, or five transduction markers could also be expressed. For example, embodiments can include a CAR construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247.

One advantage of including at least one control feature in a CAR is that cells expressing CAR administered to a subject can be increased or depleted using the cognate binding molecule to a tag cassette. In certain embodiments, the present disclosure provides a method for depleting a modified cell expressing a CAR by using an antibody specific for the tag cassette, using a cognate binding molecule specific for the control feature, or by using a second modified cell expressing a CAR and having specificity for the control feature. Elimination of modified cells may be accomplished using depletion agents specific for a control feature. For example, if tEGFR is used, then an anti-tEGFR binding domain (e.g., antibody, scFv) fused to or conjugated to a cell-toxic reagent (such as a toxin, radiometal) may be used, or an anti-tEGFR/anti-CD3 bispecific scFv, or an anti-tEGFR CAR T cell may be used.

In certain embodiments, modified cells expressing a CAR may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature with a CAR can be, e.g., more readily identified, isolated, sorted, induced to proliferate, tracked, and/or eliminated as compared to a modified cell without a tag cassette.

(iii-b-vii) Multimerization Domains. Multimerization Domains. In particular embodiments, the CAR can optionally include a multimerization domain. A “multimerization domain” is a domain that causes two or more proteins (monomers) to interact with each other through covalent and/or non-covalent association(s). Multimerization domains present in proteins can result in protein interactions that form dimers, trimers, tetramers, pentamers, hexamers, heptamers, etc., depending on the number of units/monomers incorporated into the multimer.

In particular embodiments, the multimerization domain is a dimerization domain that allows binding of two complementary monomers to form a dimer. In particular embodiments, a dimerization and docking domain (DDD) can be derived from the cAMP-dependent protein kinase (PKA) regulatory subunits and can be paired with an anchoring domain (AD). The AD can be derived from a specific region found in various A-kinase anchoring proteins (AKAPs) that mediates association with the R subunits of PKA. Additional DDDs and ADs include: the 4-helix bundle type DDD (Newlon, et al. EMBO J. 2001; 20: 1651-1662; Newlon, et al. Nature Struct Biol. 1999; 3: 222-227) domains obtained from p53, DCoH (pterin 4 a carbinolamine dehydratase/dimerization cofactor of hepatocyte nuclear factor 1 α (TCF1)) and HNF-1 (hepatocyte nuclear factor 1) (Rose, et al. Nature Struct Biol. 2000; 7: 744-748). Other AD sequences of potential use may be found in US 2003/0232420A1.

In particular embodiments, complementary binding domains can dimerize. In particular embodiments, the binding domain is a transmembrane polypeptide derived from a FcεRI chain. In particular embodiments, a CAR can include a part of a FcεRI α chain and another CAR can include a part of an FcεRI β chain such that said FcεRI chains spontaneously dimerize together to form a dimeric CAR. In particular embodiments, CAR can include a part of a FcεRI α chain and a part of a FcεRI γ chain such that said FcεRI chains spontaneously trimerize together to form a trimeric CAR, and in another embodiment the multi-chain CAR can include a part of FcεRI α chain, a part of FcεRI β chain and a part of FcεRI γ chain such that said FcεRI chains spontaneously tetramerize together to form a tetrameric CAR.

Leucine zippers are described in U.S. Pat. No. 5,932,448; SH2 and SH3 are described in Vidal et al., Biochemistry, 43:7336-44, 2004); PTB is described in Zhou et al., Nature, 378:584-592, 1995); WW is described in Sudol Prog Biochys MoL Bio, 65:113-132, 1996; PDZ is described in Kim et al., Nature, 378: 85-88, 1995 and Komau et al., Science, 269:1737-1740, 1995; and WD40 is described in Hu et al., J Biol Chem., 273:33489-33494, 1998.

Additional multimerization domains and systems are described in, for example, Hodneland, et al. Proc Natl Acd Sci USA. 2002; 99: 5048-5052; Arakawa et al., J Biol. Chem., 269:27833-27839, 1994; Radziejewski et al., Biochem, 32: 1350, 1993; WO2012001647A2; U.S. Pat. No. 5,821,333; GenBank Accession no. AAF73912.1 (Nishi et al., Mol Cell Biol, 25: 2607-2621, 2005), the SH3 domain of IB1 from GenBank Accession no. AAD22543.1 (Kristensen el al., EMBO J., 25: 785-797, 2006), the PTB domain of human DOK-7 from GenBank Accession no. NP_005535.1 (Wagner et al., Cold Spring Harb Perspect Biol. 5: a008987, 2013), the PDZ-like domain of SATB1 from UniProt Accession No. Q01826 (Galande et al., Mol Cell Biol. August; 21: 5591-5604, 2001), the WD40 repeats of APAF from UniProt Accession No. 014727 (Jorgensen et al., 2009. PLOS One. 4(12):e8463), the PAS motif of the dioxin receptor from UniProt Accession No. 16L9E7 (Pongratz et al., Mol Cell Biol, 18:4079-4088, 1998) and the EF hand motif of parvalbumin from UniProt Accession No. P20472 (Jamalian et al., Int J Proteomics, 2014: 153712, 2014). C4b, dextrameric, and ferritin-based multimerization can be used.

In particular embodiments, complementary binding domains can be induced using a third molecule or chemical inducer. This method of dimerization requires that one CAR include a chemical inducer of dimerization binding domain 1 (CBD1) and the second CAR include the second chemical inducer of dimerization binding domain (CBD2), wherein CBD1 and CBD2 are capable of simultaneously binding to a chemical inducer of dimerization (CID). CBD1 may include a rapamycin binding domain of FK-binding protein 12 (FKBP12) and CBD2 may include a FKBP12-Rapamycin Binding (FRB) domain of mTOR.

### (iv) CELL ACTIVATING CULTURE CONDITIONS

Cell populations can be incubated in a culture-initiating media to expand genetically modified cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

Exemplary culture media for culturing T cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10-4-0.75×10-4 M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T cell culture media are also commercially available from Hyclone (Logan, UT). Additional T cell activating components that can be added to such culture media are described in more detail below.

In some embodiments, the T cells are expanded by adding to the culture-initiating media feeder cells, such as AML cells, (e.g., such that the resulting population of cells contains at least 5, 10, 20, or 40 or more AML feeder cells for each T lymphocyte in the initial population to be expanded); and incubating the culture (e.g., for a time sufficient to expand the numbers of T cells).

In some aspects, the non-dividing feeder cells can include gamma-irradiated AML feeder cells. In some embodiments, the AML cells are irradiated with gamma rays in the range of 3000 to 3600 rads to prevent cell division. In some aspects, the feeder cells are added to culture medium prior to the addition of the populations of T cells. In particular embodiments, a time sufficient to expand the numbers of T cells includes 24 hours. In particular embodiments, the ratio of T cells to feeder cells is 1:1, 2:1, or 1:2.

In some embodiments, the stimulating conditions include temperature suitable for the growth of human T lymphocytes, for example, at least 25° C., at least 30° C., or 37° C.

The activating culture conditions for T cells include conditions whereby T cells of the culture-initiating media proliferate or expand. T cell activating conditions can include one or more cytokines, for example, interleukin (IL)-2, IL-7, IL-15 and/or IL-21. IL-2 can be included at a range of 10-100 ng/ml (e.g., 40, 50, or 60 ng/ml). IL-7, IL-15, and/or IL-21 can be individually included at a range of 0.1-50 ng/ml (e.g., 5, 10, or 15 ng/ml).

In particular embodiments, T cell activating culture condition conditions can include T cell stimulating epitopes. T cell stimulating epitopes include CD3, CD27, CD2, CD4, CD5, CD7, CD8, CD28, CD30, CD40, CD56, CD83, CD90, CD95, 4-1BB (CD 137), B7-H3, CTLA-4, Frizzled-1 (FZD1), FZD2, FZD3, FZD4, FZD5, FZD6, FZD7, FZD8, FZD9, FZD10, HVEM, ICOS, IL-1R, LAT, LFA-1, LIGHT, MHCI, MHCII, NKG2D, OX40, ROR2 and RTK.

In particular embodiments, a T-cell activating culture media includes a FACS-sorted T cell population cultured within RPMI with HEPES, 5-15% human serum, 1-3% L-Glutamine, 0.5-1.5% Pen/strep, 0.25×10⁻⁴-0.75×10⁻⁴ M β-MercaptoEthanol, with IL-7, IL-15 and IL-21 individually included at 5-15 (e.g., 10) ng/ml. The culture is carried out on a flat-bottom well plate with 0.1-0.5×10⁶ plated cells/well. On Day 3 post activation cells are transferred to a tissue culture (TC)-treated plate.

In particular embodiments, a T-cell activating culture media includes a FACS-sorted CD8+T population cultured within RPMI with HEPES, 10% human serum, 2% L-Glutamine, 1% Pen/strep, 0.5×10⁻⁴ M β-MercaptoEthanol, with IL-7, IL-15 and IL-21 individually included at 5-15 (e.g., 10) ng/ml. The culture is carried out on a flat-bottom non-tissue culture-treated 96/48-well plate with 0.1-0.5×10⁶ plated cells/well. On Day 3 post activation cells are transferred to TC-treated plate. Culture conditions for HSC/HSP can include expansion with a Notch agonist (see, e.g., U.S. Pat. Nos. 7,399,633; 5,780,300; 5,648,464; 5,849,869; and 5,856,441 and growth factors present in the culture condition as follows: 25-300 ng/ml SCF, 25-300 ng/ml Flt-3L, 25-100 ng/ml TPO, 25-100 ng/ml IL-6 and 10 ng/ml IL-3. In more specific embodiments, 50, 100, or 200 ng/ml SCF; 50, 100, or 200 ng/ml of Flt-3L; 50 or 100 ng/ml TPO; 50 or 100 ng/ml IL-6; and 10 ng/ml IL-3 can be used.

### (v) EX VIVO MANUFACTURED CELL FORMULATIONS

In particular embodiments, genetically modified cells can be harvested from a culture medium and washed and concentrated into a carrier in a therapeutically-effective amount. Herein, cell formulations refers to the formulations including cells genetically modified to express a CAR disclosed herein and prepared for administration. Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions and/or formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions and/or formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

In particular embodiments, formulations include at least one genetically modified cell type (e.g., modified T cells, NK cells, or stem cells). Formulations can include different types of genetically-modified cells (e.g., T cells, NK cells, and/or stem cells in combination).

Different types of genetically-modified cells or cell subsets (e.g., modified T cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different CAR components.

The cell-based formulations disclosed herein can be prepared for administration by, e.g., injection, infusion, perfusion, or lavage. The formulations and formulations can further be formulated for bone marrow, intravenous, intradermal, intraarterial, intranodal, intralymphatic, intraperitoneal, intralesional, intratumoral, intravesicular, and/or subcutaneous injection.

### (vi) COMPOSITIONS

Compositions include (i) immune-cell targeted nanoparticles and/or immune-cell targeted vectors (collectively referred to as “active ingredients” hereafter) that result in in vivo modification of the targeted immune cell to express a CAR disclosed herein and (ii) a pharmaceutically acceptable carrier. Any of the active ingredients described herein in any exemplary format or conjugation form can be formulated alone or in combination into compositions for administration to subjects. Salts and/or pro-drugs of the active ingredients can also be used.

A pharmaceutically acceptable salt includes any salt that retains the activity of the active ingredients and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles. Exemplary carriers include saline, buffered saline, physiological saline, water, Hanks' solution, Ringer's solution, Nonnosol-R (Abbott Labs), Plasma-Lyte A® (Baxter Laboratories, Inc., Morton Grove, IL), glycerol, ethanol, and combinations thereof.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas.

Additionally, compositions can be formulated as sustained-release systems utilizing semipermeable matrices of solid polymers including at least one type of antibody conjugate or nanoparticle.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

### (vii) METHODS OF USE

Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with formulations and/or compositions disclosed herein. Treating subjects includes delivering therapeutically effective amounts.

Therapeutically effective amounts include those that provide effective amounts, prophylactic treatments and/or therapeutic treatments.

An “effective amount” is the amount of a formulation and/or composition necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer's development or progression. An immunogenic formulation can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a PRAME-expressing cancer.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, prolonged subject life, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

In particular embodiments, therapeutically effective amounts induce an immune response. The immune response can be against a cancer cell.

In particular embodiments, the cancer is acute myeloid leukemia (AML). In particular embodiments, the cancer is t(8;21) AML, Inv(16) AML, or KMT2A-r AML. In particular embodiments, the cancer is breast cancer, sarcoma, or neuroblastoma. In particular embodiments, the cancer is another PRAME-expressing cancer, such as medulloblastoma, hepatocellular carcinoma, adenocarcinoma, uveal melanoma, high-grade serous cancer, myxoid liposarcoma, diffuse large B-cell lymphoma, osteosarcoma, bladder cancer, melanoma, breast cancer, neuroblastoma, ovarian cancer, cervical cancer, lung cancer, or a hematologic malignancy.

Formulations and/or compositions disclosed herein can also be used to treat a complication or disease related to AML. For example, complications relating to AML may include a preceding myelodysplastic syndrome (MDS, formerly known as “preleukemia”), secondary leukemia, in particular secondary AML, high white blood cell count, and absence of Auer rods.

Among others, leukostasis and involvement of the central nervous system (CNS), hyperleukocytosis, residual disease, are also considered complications or diseases related to AML.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of cancer, previous or concurrent therapeutic interventions, idiopathy of the subject and route of administration.

Therapeutically effective amounts of cell-based formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of compositions can include 0.1 μg/kg to 5 mg/kg body weight, 0.5 μg/kg to 2 mg/kg, or 1 mg/kg to 4 mg/kg. Therapeutically effective amounts to administer can include greater than 0.1 μg/kg, greater than 0.6 μg/kg, greater than 1 mg/kg, greater than 2 mg/kg, greater than 3 mg/kg, greater than 4 mg/kg, or greater than 5 mg/kg.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intrathecal, intratumoral, intravesicular, and/or subcutaneous.

In certain embodiments, formulations and/or compositions are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

### (viii) EXEMPLARY EMBODIMENTS

1. A chimeric antigen receptor (CAR) that, when expressed by a cell, includes an extracellular component linked to an intracellular component through a transmembrane domain, wherein the extracellular component includes a Preferentially Expressed Antigen in Melanoma (PRAME) ALY/HLA-A2 binding domain and the intracellular component includes an effector domain.
2. The CAR of embodiment 1, wherein the binding domain includes a single chain variable fragment (scFv).
3. The CAR of embodiment 1 or 2, wherein the scFv has at least 90% sequence identity to the sequence as set forth in SEQ ID NO: 43 and/or SEQ ID NO: 44.
4. The CAR of embodiment 2 or 3, wherein the scFv has the sequence as set forth in SEQ ID NO: 43 or SEQ ID NO: 44.
5. The CAR of any of embodiments 2-4, wherein the scFv is encoded by a sequence having at least 90% sequence identity to the sequence as set forth in SEQ ID NO: 3 and or SEQ ID NO: 4.
6. The CAR of any of embodiments 2-5, wherein the scFv is encoded by the sequence as set forth in SEQ ID NO: 3 or SEQ ID NO: 4.
7. The CAR of any of embodiments 1-6, wherein the binding domain includes a variable light chain including at least 90% sequence identity to a sequence as set forth in SEQ ID NO: 41 and a variable heavy chain including a sequence as set forth in SEQ ID NO: 42.
8. The CAR of any of embodiments 1-7, wherein the binding domain includes a variable light chain including a sequence as set forth in SEQ ID NO: 41 and a variable heavy chain including a sequence as set forth in SEQ ID NO: 42.
9. The CAR of any of embodiments 1-8, wherein the binding domain includes a variable light chain encoded by a sequence having at least 90% sequence identity to the sequence as set forth in SEQ ID NO: 1 and a variable heavy chain encoded by a sequence having at least 90% sequence identity to the sequence as set forth in SEQ ID NO: 2.
10. The CAR of any of embodiments 1-9, wherein the binding domain includes a variable light chain encoded by the sequence as set forth in SEQ ID NO: 1 and a variable heavy chain encoded by the sequence as set forth in SEQ ID NO: 2.
11. The CAR of any of embodiments 1-10, wherein the binding domain includes a variable light chain complementarity determining region (CDRL) 1 as set forth in SEQ ID NO: 48, a CDRL2 as set forth in SEQ ID NO: 49, and a CDRL3 as set forth in SEQ ID NO: 50 and a variable heavy chain with complementarity determining regions (CDRH) 1 as set forth in SEQ ID NO: 45, a CDRH2 as set forth in SEQ ID NO: 46, and a CDRH3 as set forth in SEQ ID NO: 47;
a CDRL1 as set forth in SEQ ID NO: 48, a CDRL2 as set forth in SEQ ID NO: 49, a CDRL3 as set forth in SEQ ID NO: 50, a CDRH1 as set forth in SEQ ID NO: 51, a CDRH2 as set forth in SEQ ID NO: 52, and a CDRH3 as set forth in SEQ ID NO: 53;
a CDRL1 as set forth in SEQ ID NO: 56, a CDRL2 including the sequence SNN, a CDRL3 as set forth in SEQ ID NO: 50, a CDRH1 as set forth in SEQ ID NO: 54, a CDRH2 as set forth in SEQ ID NO: 55, and a CDRH3 as set forth in SEQ ID NO: 47;
a CDRL1 as set forth in SEQ ID NO: 48, a CDRL2 as set forth in SEQ ID NO: 58, a CDRL3 as set forth in SEQ ID NO: 50, a CDRH1 as set forth in SEQ ID NO: 54, a CDRH2 as set forth in SEQ ID NO: 57, and a CDRH3 as set forth in SEQ ID NO: 53; or
a CDRL1 as set forth in SEQ ID NO: 62, a CDRL2 as set forth in SEQ ID NO: 63, a CDRL3 as set forth in SEQ ID NO: 64, a CDRH1 as set forth in SEQ ID NO: 59, a CDRH2 as set forth in SEQ ID NO: 60, and a CDRH3 as set forth in SEQ ID NO: 61.
   12. The CAR of any of embodiments 1-11, wherein the extracellular component further includes a spacer region.
   13. The CAR of embodiment 12, wherein the spacer region is less than 50 amino acids and consists of an IgG4 hinge.
   14. The CAR of embodiment 13, wherein the IgG4 hinge includes an IgG4 hinge with S10P mutation.
   15. The CAR of any of embodiments 12-14, wherein the spacer region is encoded by a sequence having at least 90% sequence identity to the sequence as set forth in SEQ ID NO: 16 and/or SEQ ID NO: 17.
   16. The CAR of any of embodiments 12-15, wherein the spacer region is encoded by the sequence as set forth in SEQ ID NO: 16 or SEQ ID NO: 17.
   17. The CAR of any of embodiments 1-16, wherein the intracellular effector domain includes all or a portion of the signaling domain of CD3ζ and 4-1BB.
   18. The CAR of embodiment 17, wherein the CD3ζ signaling domain includes a sequence having at least 90% sequence identity to the sequence as set forth in SEQ ID NO: 20 and/or SEQ ID NO: 21.
   19. The CAR of embodiment 17 or 18, wherein the CD3ζ signaling domain includes the sequence as set forth in SEQ ID NO: 20 or SEQ ID NO: 21.
   20. The CAR of any of embodiments 17-19, wherein the CD3ζ signaling domain is encoded by a sequence having at least 90% sequence identity to the sequence as set forth in SEQ ID NO: 22.
   21. The CAR of any of embodiments 17-20, wherein the CD3ζ signaling domain is encoded by the CD3ζ coding sequence as set forth in SEQ ID NO: 22.
   22. The CAR of any of embodiments 17-21, wherein the 4-1BB signaling domain includes a sequence having at least 90% sequence identity to the sequence as set forth in SEQ ID NO: 23 and/or SEQ ID NO: 24.
   23. The CAR of any of embodiments 17-22, wherein the 4-1BB signaling domain includes the sequence as set forth in SEQ ID NO: 23 or SEQ ID NO: 24.
   24. The CAR of any of embodiments 17-23, wherein the 4-1 BB signaling domain is encoded by a sequence having at least 90% sequence identity to the sequence as set forth in SEQ ID NO: 25 and/or SEQ ID NO: 26.
   25. The CAR of any of embodiments 17-24, wherein the 4-1 BB signaling domain is encoded by the sequence as set forth in SEQ ID NO: 25 or SEQ ID NO: 26.
   26. The CAR of any of embodiments 1-25, wherein the transmembrane domain includes a CD28 transmembrane domain.
   27. The CAR of embodiment 26, wherein the CD28 transmembrane domain includes a sequence having at least 90% sequence identity to the sequence as set forth in SEQ ID NO: 27 and/or SEQ ID NO: 28.
   28. The CAR of embodiment 26 or 27, wherein the CD28 transmembrane domain includes the sequence as set forth in SEQ ID NO: 27 or SEQ ID NO: 28.
   29. The CAR of any of embodiments 26-28, wherein the CD28 transmembrane domain is encoded by a sequence having at least 90% sequence identity to the sequence as set forth in SEQ ID NO: 29, SEQ ID NO: 30, and/or SEQ ID NO: 31.
   30. The CAR of any of embodiments 26-29, wherein the CD28 transmembrane domain is encoded by the sequence as set forth in SEQ ID NO: 29, SEQ ID NO: 30, or SEQ ID NO: 31.
   31. The CAR of any of embodiments 1-30, further including a control feature selected from a tag cassette, a transduction marker, and/or a suicide switch.
   32. The CAR of embodiment 31, wherein the transduction marker includes a truncated CD19.
   33. The CAR of embodiment 32, wherein the truncated CD19 includes a sequence having at least 90% sequence identity to the sequence as set forth in SEQ ID NO: 38.
   34. The CAR of embodiment 32 or 33, wherein the truncated CD19 includes the sequence as set forth in SEQ ID NO: 38.
   35. The CAR of any of embodiments 32-34, wherein the truncated CD19 is encoded by a sequence having at least 90% sequence identity to the sequence as set forth in SEQ ID NO: 39.
   36. The CAR of any of embodiments 32-35, wherein the truncated CD19 is encoded by the sequence as set forth in SEQ ID NO: 39.
   37. The CAR of any of embodiments 1-36, further including a ribosomal skip element.
   38. The CAR of embodiment 37, wherein the ribosomal skip element includes T2A, P2A, E2A, or F2A.
   39. The CAR of embodiment 37 or 38, wherein the ribosomal skip element includes T2A.
   40. The CAR of any of embodiments 1-39, including a sequence having at least 90% sequence identity to the sequence as set forth in SEQ ID NO: 5, SEQ ID NO: 7, and/or SEQ ID NO: 9.
   41. The CAR of any of embodiments 1-40, including the sequence as set forth in SEQ ID NO: 5, SEQ ID NO: 7, or SEQ ID NO: 9.
   42. The CAR of any of embodiments 1-41, encoded by a sequence having at least 90% sequence identity to the sequence as set forth in SEQ ID NO: 6, SEQ ID NO: 8, SEQ ID NO: 10, or SEQ ID NO: 11.
   43. The CAR of any of embodiments 1-42, encoded by the sequence as set forth in SEQ ID NO: 6, SEQ ID NO: 8, SEQ ID NO: 10, and/or SEQ ID NO: 11.
   44. A genetic construct encoding the CAR of any of embodiments 1-43.
   45. The genetic construct of embodiment 44, including a sequence having at least 90% sequence identity to the sequence as set forth in SEQ ID NO: 11.
   46. The genetic construct of embodiment 44 or 45, including the sequence as set forth in SEQ ID NO: 11.
   47. A nanoparticle encapsulating the genetic construct of any of embodiments 44-46.
   48. A cell including a genetic construct that results in expression of the CAR of any of embodiments 1-43 by the cell.
   49. The cell of embodiment 48, wherein the cell is in vivo or ex vivo.
   50. The cell of embodiment 48 or 49, wherein the cell is a T cell, B cell, natural killer (NK) cell, NK-T cell, monocyte/macrophage, hematopoietic stem cells (HSC), or a hematopoietic progenitor cell (HPC).
   51. The cell of any of embodiments 48-50, wherein the cell is a T cell selected from a CD3+ T cell, a CD4+ T cell, a CD8+ T cell, a central memory T cell, an effector memory T cell, and/or a naïve T cell.
   52. The cell of any of embodiments 48-51, wherein the cell is a CD8+ T cell and/or a CD4+ T cell.
   53. A formulation including the cell of any of embodiments 48-52 and a pharmaceutically acceptable carrier.
   54. The formulation of embodiment 53, wherein the cells are T cells, natural killer cells, monocyte/macrophages, hematopoietic stem cells or hematopoietic progenitor cells.
   55. The formulation of embodiment 54, wherein the T cells are selected from CD3 T cells, CD4 T cells, CD8 T cells, central memory T cells, effector memory T cells, and/or naïve T cells.
   56. The formulation of embodiment 54 or 55, wherein the T cells are CD4 T cells and/or CD8 T cells.
   57. A composition including (i) an immune-cell targeted nanoparticle and/or an immune-cell targeted vector including a genetic construct that results in expression of the CAR of any of embodiments 1-43 by the immune cell following transfection of the immune cell and (ii) a pharmaceutically acceptable carrier.
   58. A method of treating a subject with a PRAME ALY/HLA-A2 expressing cancer, the method including administering a therapeutically effective amount of the formulation of any of embodiments 53-56 and/or the composition of embodiment 57 to the subject thereby treating the subject with the PRAME ALY/HLA-A2 expressing cancer.
   59. The method of embodiment 58, wherein the PRAME ALY/HLA-A2 expressing cancer is acute myeloid leukemia (AML).
   60. The method of embodiment 59, wherein the AML is t(8;21) AML, Inv(16) AML, or KMT2A-r AML.
   61. The method of any of embodiments 58-60, further including screening the subject for the PRAME ALY/HLA-A2 expressing cancer.
   62. The method of embodiment 61, further including selecting the subject for treatment based on the screening.
   63. The method of any of embodiments 58-62, further including administering interferon gamma (IFNγ) to the subject.
   64. The method of any of embodiments 58-63, wherein the formulation includes autologous cells or allogeneic cells in reference to the subject.

### (ix) EXPERIMENTAL EXAMPLE

Preferentially Expressed Antigen in Melanoma (PRAME), a cancer testes antigen provides an ideal target for immunotherapy in AML. Expression of PRAME in a significant subset of childhood and adult AML and lack of expression in normal hematopoiesis is shown. Although an intracellular antigen, a novel approach to target PRAME using a CAR construct encoding a binding domain based on T cell receptor (TCR) mimic antibodies that binds the peptide:HLA complex was developed. The Pr20 antibody sequence was used. This antibody is a TCR mimic antibody that recognizes PRAME ALY peptide in complex with HLA-A*02. Expression of PRAME in AML cell lines and primary AML blasts was also verified. Using the Pr20 antibody sequence, CAR T cells (PRAME ^{mTCR}CAR T) were tested against primary AML patient samples and AML cell lines that express the PRAME antigen in the context of HLA-A2 expression. In contrast to the appropriate controls, PRAME ^{mTCR}CAR T cells demonstrate target specific and HLA-mediated in vitro activity in OCI-AML2 and THP-1 cell lines, HLA-A2 cell lines expressing the PRAME antigen, and against primary AML patient samples. In vivo cell-derived xenograft models treated with PRAME ^{mTCR}CAR T cells demonstrated potent leukemia clearance and improved survival compared to unmodified T cell controls. Furthermore, the cytolytic activity of PRAME ^{mTCR}CAR T cells was enhanced by treating the target cells with IFN-gamma, which increases PRAME antigen expression. These results demonstrate the efficacy of targeting PRAME with novel PRAME ^{mTCR}CAR T cells.

Materials and Methods. Generation of PRAME CAR constructs. CAR constructs containing an IgG4 hinge and 41-BB/CD3 signaling domain as described in Turtle et al., J Clin Invest. 2016, 126(6):2123-2138 were selected for further development. The variable light (VL) and heavy (VH) sequences from Pr20 antibody (Chang et al., J Clin Invest. 2017, 127(9):3557) were used to construct the single-chain fragment variable domain of the 41BB/CD3 CAR vector.

Generation of human PRAME ^{mTCR}CAR T cells. CAR T cells were generated by transducing healthy donor T cells (Bloodworks Northwest) with lentivirus carrying the CAR vector under the approval of FHCC Institutional Review Board (protocol 5608). Peripheral blood mononuclear cells from healthy donors were isolated over Lymphoprep (StemCell Technologies, catalog no. 07851). CD4 or CD8 T cells were isolated by negative magnetic selection using Easy Sep Human CD4+ T cell Isolation Kit II (StemCell Technologies, catalog no. 17952) and Easy Sep Human CD8+ T cell Isolation Kit II (StemCell Technologies, catalog no. 17953). Purified T cells were cultured in CTL media [RPMI supplemented with 10% human serum (Bloodworks Northwest), 2% L-glutamine (Gibco, catalog no. 25030-081), 1% penicillin-streptomycin (Gibco, catalog no. 15140-122), 0.5 mol/L β-mercaptoethanol (Gibco, catalog no. 21985-023), and 50 U/mL IL2 (aldesleukin, Prometheus)] at 37° C. in 5% CO₂. T cells were activated with anti-CD3/CD28 beads (3:1 beads: cell, Gibco, 11131D) on Retronectin-coated plates (5 μg/mL, coated overnight at 4° C.; Takara, catalog no. T100B) and transduced with CAR lentivirus (MOI=50) one day after activation via spinoculation at 800×g for 90 minutes at 25° C. in CTL media (+50 U/mL IL2) supplemented with 8 μg/mL protamine sulfate. Transduction used 200,000 cells/well in 24-well plates. Transduced cells were expanded in CTL media (+50 U/mL IL2) and separated from beads on day 5. Truncated CD19 was co-expressed with the CAR by a T2A ribosomal skip element to select for transduced cells, which were sorted for CD19 expression [using anti-human CD19 PE (BioLegend, catalog no. 982402)] on FACSAria II 8-10 days post-activation. Sorted cells were further expanded in CTL (+50 U/mL IL2) media prior to in vitro and in vivo cytotoxicity assays.

Cell lines. OCI-AML2, THP1, K562, MV4;11 and RS4;11 cells were obtained from ATCC and maintained per the manufacturer's instructions. MV4;11 and RS4:11 cells were transduced with an HLA-A2 expression construct to generate MV4;11 HLA-A2+ and RS4:11 HLA-A2+ cell lines.

Primary samples. Frozen aliquots of mononuclear CD45+ cells isolated from AML diagnostic bone marrow samples were obtained from the Children's Oncology Group. Freshly thawed aliquots were assessed for PRAME by flow cytometry. Some samples were sent to Hematologics, Inc. for immunophenotype analysis of PRAME expression. All specimens used in this example were obtained after written content from patients and donors. The research was performed after approval by the FHCC Institutional Review Board (protocol #5608). The study was conducted in accordance with the Declaration of Helsinki.

In vitro studies. Target cells (OCI-AML2, THP1, and K562 for AML studies or SK-N-SH parental or HLA-A2 transduced for neuroblastoma studies) were split 1-2 days prior to cytotoxicity assays. Target cells were labeled with 2.5 μmol/L carboxyfluorescein succinimidyl ester (CFSE) (Invitrogen, catalog no. C34554) per the manufacturer's directions, washed with 1×PBS, and resuspended in CTL media (without IL2). For T-cell proliferation assays, effector cells (unmodified or PRAME ^{mTCR}CAR T cells) were labeled with 2.5 mol/L Violet Cell Proliferation Dye (Invitrogen, catalog no. C34557) washed with 1×PBS, serial diluted in CTL media (without IL2), and combined with target cells at various effector:target (E:T) ratios in 96 U-bottom plate. Cytotoxicity was assessed by flow cytometry after staining cells with live/dead fixable viability dyes [FVD; Invitrogen, catalog no. L34964]. Percent dead among target cells was assessed by gating on FVD⁺ among CFSE⁺ target cells. For primary patient samples, percent live cells were assessed by gating on FVD⁻ among CFSE⁺ target cells. Percent-specific lysis was calculated by subtracting the average of the three replicate wells containing target cells only from each well containing target and effector cells at each E:T ratio. After 24 hours of coculture, media supernatant was assessed for IL2, IFNγ, and TNFα production by Luminex microbead technology (provided by FHCC Immune Monitoring Core).

In vivo studies. For cell line-derived xenograft (CDX) models, OCI-AML2, THP-1 and K562 cells were transduced with green fluorescent protein (GFP)/luciferase construct (Plasmid #104834, Addgene) and sorted for GFP+ cells. Luciferase-expressing cells were injected intravenously into NSG mice through the tail vein at 1×10⁶ cells per mouse. Mice were treated with 5×10⁶ cells (1:1 CD4:CD8) per mouse of either PRAME ^{mTCR}CAR or unmodified T cells via tail vein intravenous injection 7 days following OCI-AML2, THP-1 and K562 injection. Leukemia burden was measured by bioluminescence imaging weekly. Leukemia burden and T-cell expansion were monitored by flow cytometric analysis of mouse peripheral blood drawn by retro-orbital bleeds for the indicated time points starting from the first week of T-cell injection. Mice were monitored and euthanized when they exhibited symptomatic leukemia (tachypnea, hunchback, persistent weight loss, fatigue, or hind-limb paralysis). Tissues (blood, bone marrow, liver, spleen, and tumors) were harvested at necropsy and analyzed for the presence of T and leukemia cells. This study was performed after approval by FHCC IACUC (protocol #51068).

Flow cytometry of xenograft cells. PE-conjugated Pr20 antibody was used to confirm PRAME expression on target cell lines and primary patient AML samples. Cell lines and primary samples were washed in 2% FBS in PBS, blocked with 20 ug/mL Fc receptor block (BD Pharmingen, catalog no. 564219) in PBS then stained with PE-conjugated anti-human Pr20 antibody and APC-conjugated HLA-A2 antibody for 20 minutes on ice. Labeled cells were washed with PBS and resuspended in 2% FBS/PBS prior to flow cytometric analysis.

Tissues were harvested at necropsy and passed through a 70-μm cell strainer to dissociate tissues into single cells prior to antibody staining. Cells from mouse peripheral blood were processed with red blood cell lysis buffer, washed in 2% FBS in PBS, blocked with 20 ug/mL Fc receptor block (BD Pharmingen, catalog no. 564219) in PBS, then stained with a cocktail of fluorescently labeled mAbs that included a combination of APC/Cyanine 7-conjugated anti-mouse CD45.1 (BioLegend, catalog no. 110716), BUV805-conjugated anti-human CD45 (BD Biosciences, catalog no. 612891), APC-conjugated anti-human CD19 (BD Biosciences, catalog no. 555415), PE-Cy7-conjugated anti-human CD3 (BD Biosciences, catalog no. 563423), BV605-conjugated anti-human CD4 (BioLegend, catalog no. 317438), BV711-conjugated anti-human CD8 (BD Biosciences, catalog no. 563677), PerCP/Cyanine 5.5-conjugated anti-human CD33 (BioLegend, catalog no. 303414), and PE-conjugated anti-human Pr20 for 20 minutes on ice. Labeled cells were washed with PBS and resuspended in 2% FBS/PBS prior to flow cytometric analysis. Symphony with FACSDiva Software (BD Biosciences) was used to assess cell surface expressions and FlowJo Software was used for the analysis. Dead cells were excluded using DAPI staining.

In vivo patient-derived xenograft studies. The PDX model was derived from a PRAME⁺/HLA-A2⁺ pediatric patient with AML. PDX cells were transduced with lentiviral luciferase for noninvasive bioluminescent IVIS imaging to monitor leukemic progression. Mice were injected with 1×10⁶ PDX leukemia cells via tail-vein injection. PDX leukemia-bearing mice were then treated with unmodified T cells or PRAME ^{mTCR}CAR T cells at 5×10⁶ cells (1:1 CD4:CD8) per mouse 1 week following leukemia injection. Leukemia burden was measured by IVIS imaging and regular peripheral blood analysis.

Statistical analysis. Unpaired, two-tailed Student t test was used to determine statistical significance for all in vitro studies. Log-rank (Mantel-Cox) test was used to compare Kaplan-Meier survival curves between experimental groups. P values <0.05 were statistically significant.

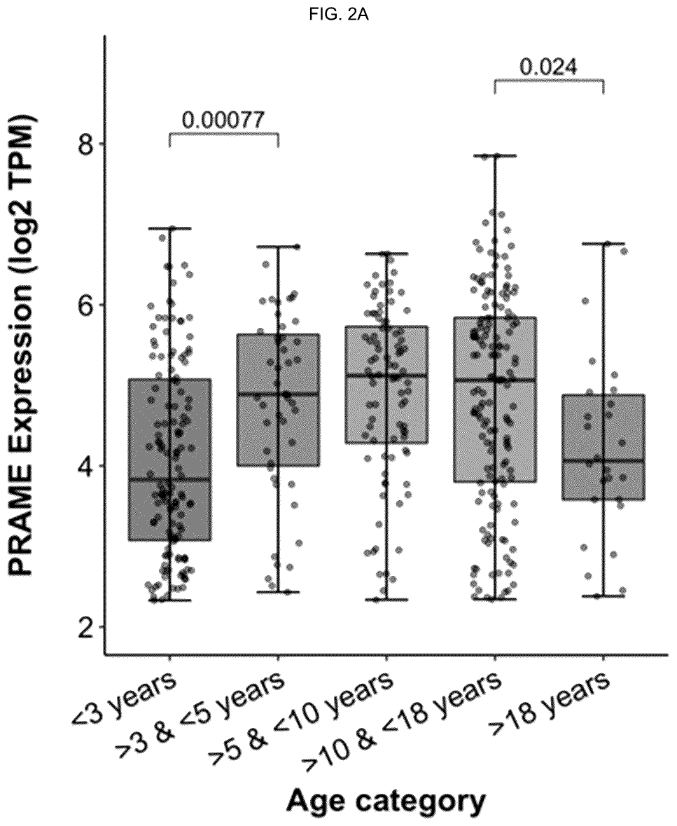

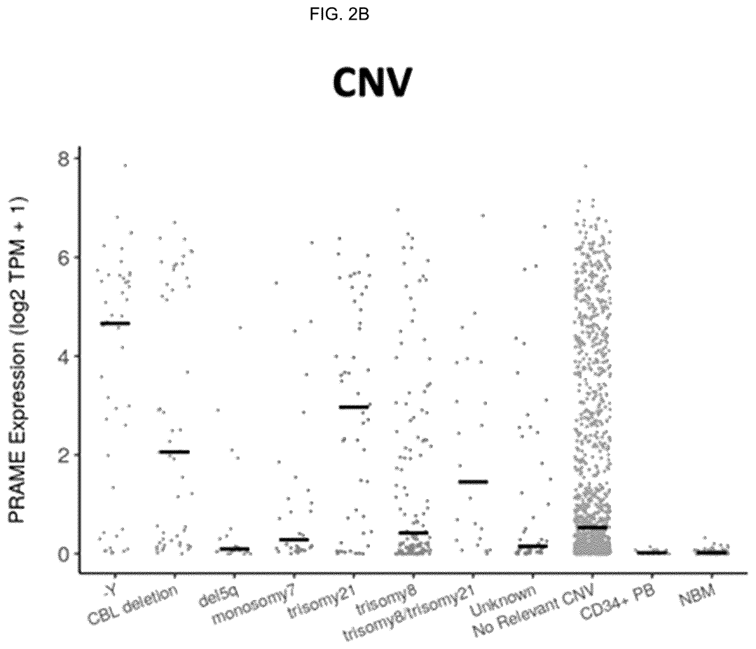

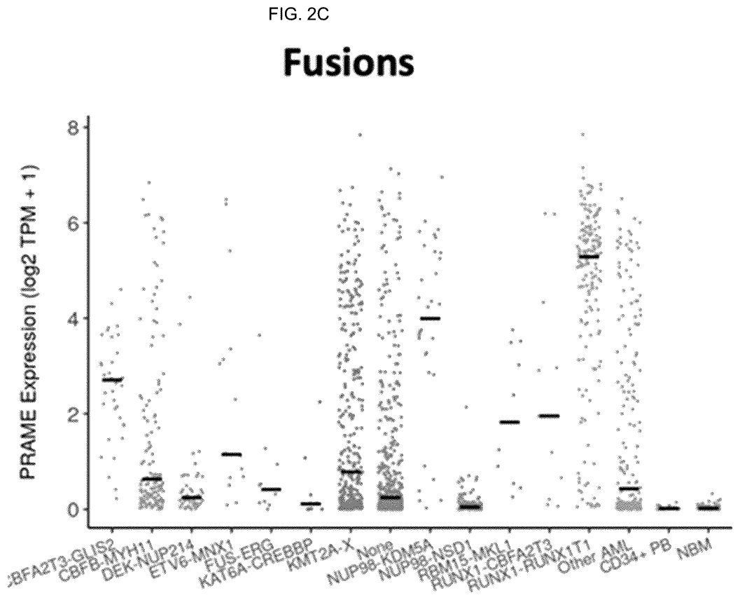

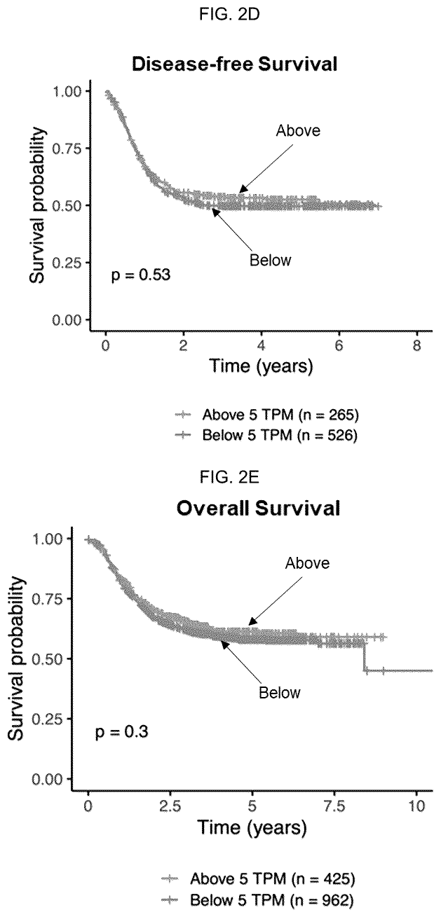

Results. PRAME transcript is expressed in AML. Analysis of the pediatric AML transcriptome (TpAML) identified PRAME to be highly expressed in a subset of leukemias without expression in normal CD34+ peripheral blood or bone marrow samples (FIG. 1A). Defining PRAME positive leukemia at TPM>5, 451 out of 1493 (30%) pediatric leukemias are positive for PRAME expression. Amongst PRAME-positive leukemias, the median expression is 24.74 TPM (range: 5.02-230.53). In adult AML (SWOG-AML), PRAME is also expressed albeit at a lower prevalence (FIG. 1B). To determine whether PRAME expression correlates with specific AML-associated molecular alterations, PRAME expression by fusion and mutation groups (FIG. 1C) were evaluated. PRAME transcript was detected in all AML subtypes, with expression in 31% of KMT2A-r AML, 23% in Inv(16) and high enrichment in t(8;21) (83% PRAME+). Furthermore, AML patients with t(8;21) had significantly higher PRAME expression compared to all other groups [median expression (range): 41.87 (5.47-230.53) for t(8;21); 24.1 (5.16-113.74) for Inv16; 19.55 (5.14-228.58) for KMT2A-r; 17.82 (5.02-139.19), other)]. PRAME expression as compared to age was evaluated and younger (<3 years; p<0.001) and older (>18 years, p=0.024) patients had lower PRAME expression, but there was no statistical difference amongst the other age groups, nor was there a correlation with outcome (FIGS. 2A-2E).

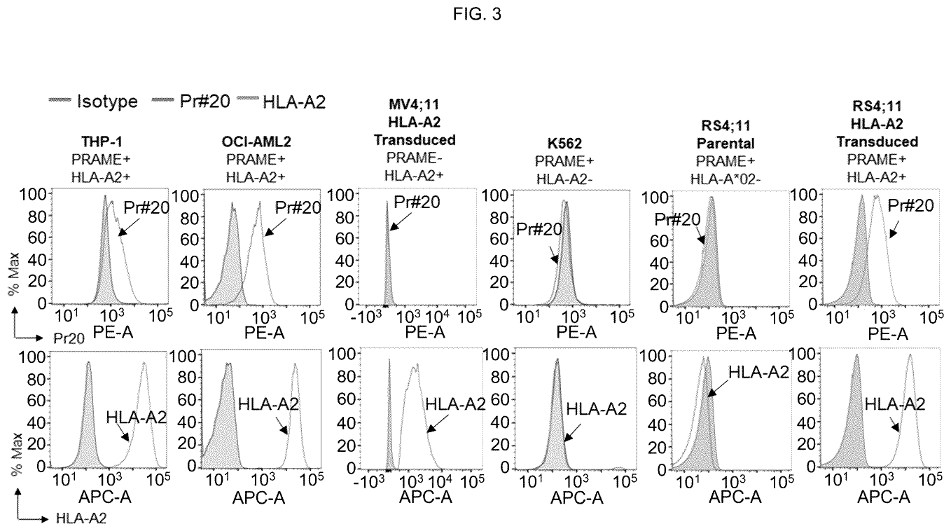

PRAME/HLA-A2 complex is expressed on the cell surface of AML blasts. A TCR mimic antibody that recognizes a specific PRAME peptide (PRAME ALYVDSLFFL (ALY) peptide (SEQ ID NO: 94) when presented in complex with HLA-A2 was developed using the Pr20 antibody (see Chang et al., J Clin Invest. 2017, 127(9):3557). The specificity of the Pr20 monoclonal antibody (mAb) against AML cell lines was verified. As expected, Pr20 bound to HLA-A2 positive THP-1 and OCI-AML2 that express PRAME (FIG. 3). Pr20 did not recognize HLA-A2 transduced MV4;11 cells, which lack PRAME expression. Pr20 also did not bind to K562 and RS4;11 cell lines, which express PRAME but not HLA-A2. However, RS411 cells transduced with an HLA-A2 expression construct were recognized by Pr20 mAb demonstrating that Pr20 specificity is dependent on PRAME expression in the context of HLA-A2. These results confirm the specificity of Pr20 against HLA-A2 positive leukemias that express PRAME.

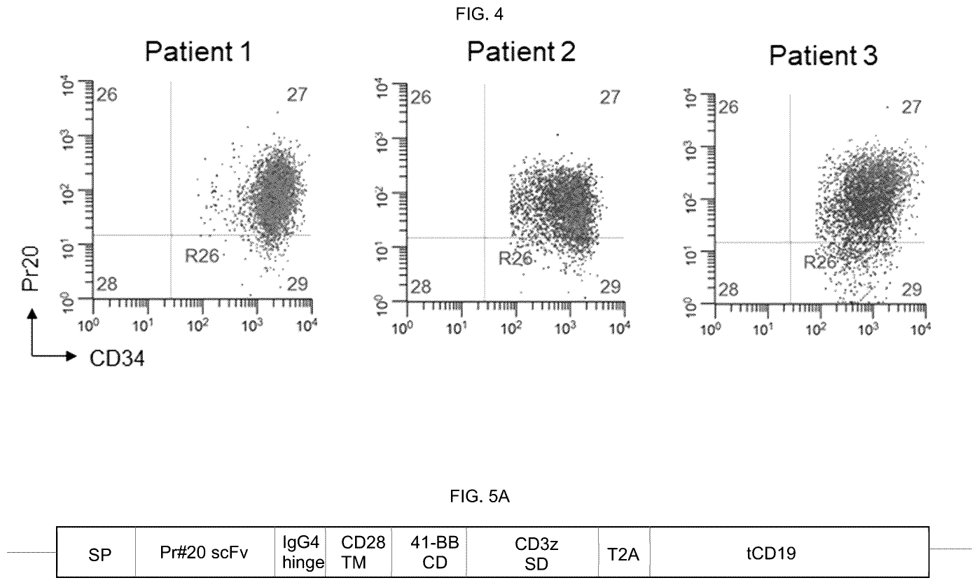

The binding of Pr20 in primary AML cells was next evaluated. In three index cases, near uniform expression of PRAME on AML blasts (FIG. 4) was detected with additional patient samples (N=224) showing cell surface PRAME antigen detected by the Pr20 antibody. Among the Pr20-positive cases (defined by expression above autofluorescence in >20% of AML blasts), PRAME antigen was moderately-to-highly expressed with median mean fluorescence intensity (MFI) of 30.6 (range: 4.4-1174.9).

PRAME ^{mTCR}CAR induce potent cytotoxicity and cytokine production in vitro. Having verified cell surface expression of PRAME in AML cell lines and patient samples, it was investigated whether PRAME positive cells can be targeted with PRAME specific CAR T cells. A Pr20 specific CAR was developed by reformatting the sequences from the Pr20 mAb into a single-chain variable fragment (scFv, see Material and Methods) and incorporating the scFv into a CAR with 41-BB costimulatory and CD3zeta signaling domains (FIG. 5A). The cytotoxicity of the CAR T cells directed at the Pr20 antigen (PRAME ^{mTCR}CAR T cells) was tested against OCI-AML2, THP-1 and K562 cells. Co-incubation of CD8 PRAME ^{mTCR}CAR T cells with OCI-AML2 and THP-1 cells for 24 hours at the indicated effector:target (E:T) ratios resulted in robust killing, whereas control, unmodified CD8 T cells did not result in cytotoxicity of the target cells (FIG. 5B, FIGS. 6A-C). Consistent with specificity, K562 cells were not susceptible to CD8 PRAME ^{mTCR}CAR T cell-mediated killing.

The cytotoxicity of the CAR T cells against primary patient AML blasts was then tested. Co-incubation of CD8 PRAME ^{mTCR}CAR T cells with PRAME+/HLA-A2+ primary patient AML blasts resulted in specific killing, most notably at the higher E:T ratios (FIG. 5C). PRAME+/HLA-A2-primary AML blasts co-incubated with CD8 PRAME ^{mTCR}CAR T cells did not demonstrate CAR T cell-mediated lysis, confirming specificity in primary patient samples (FIG. 5C). To further demonstrate the reactivity of PRAME ^{mTCR}CAR T cells, cytokine production after 24 hours of co-incubation with OCI-AML2 and THP1 cells with CD4 and CD8 PRAME ^{mTCR}CAR T cells at 1:1 E:T ratio was measured. Both CD4 and CD8 CAR T cells produced significant levels of pro-inflammatory cytokines (IL-2, IFN-gamma, and TNF-alpha) when co-incubated with OCI-AML2 and THP-1 cells (FIG. 5D, FIGS. 5B-5C) with no cytokine production noted when co-incubated with K562 cells (results not shown). Together, these results demonstrate the antigen-dependent reactivity of PRAME ^{mTCR}CAR T cells against AML cell lines and primary AML patient samples expressing PRAME.

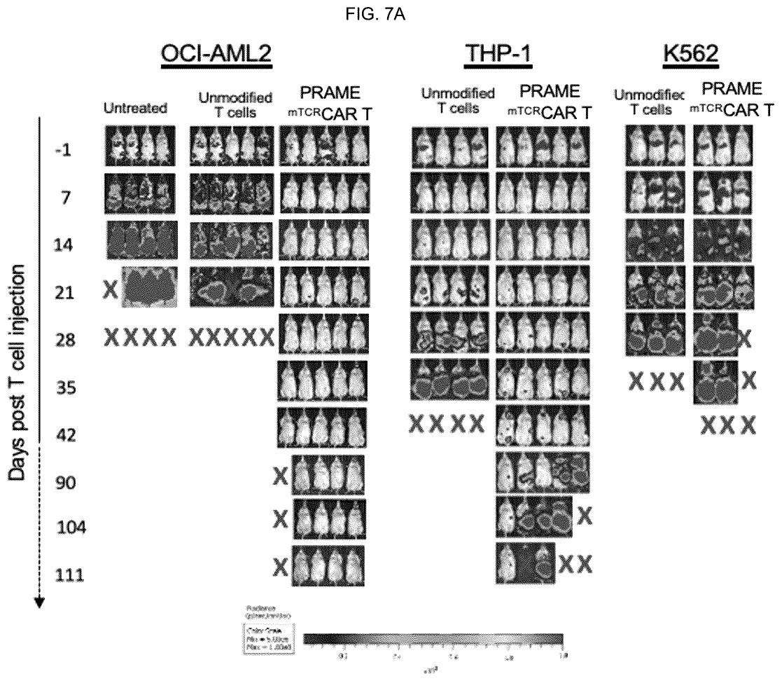

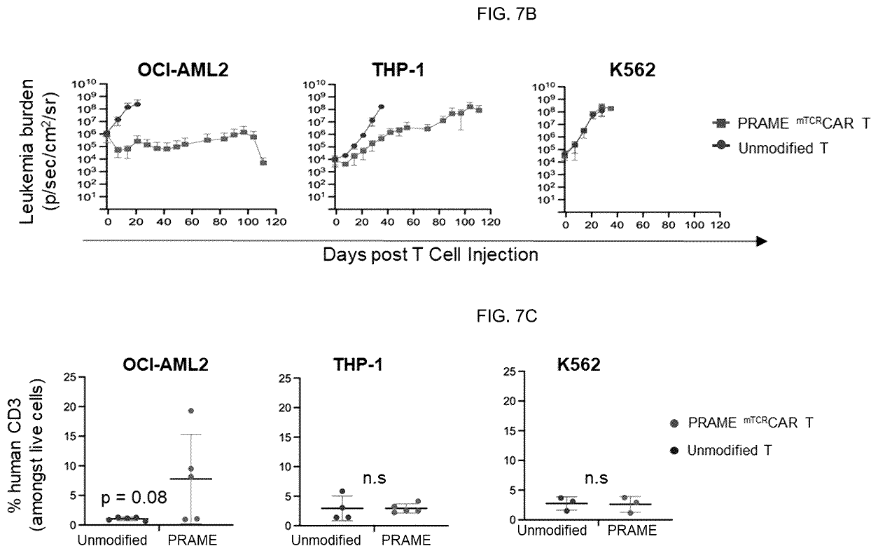

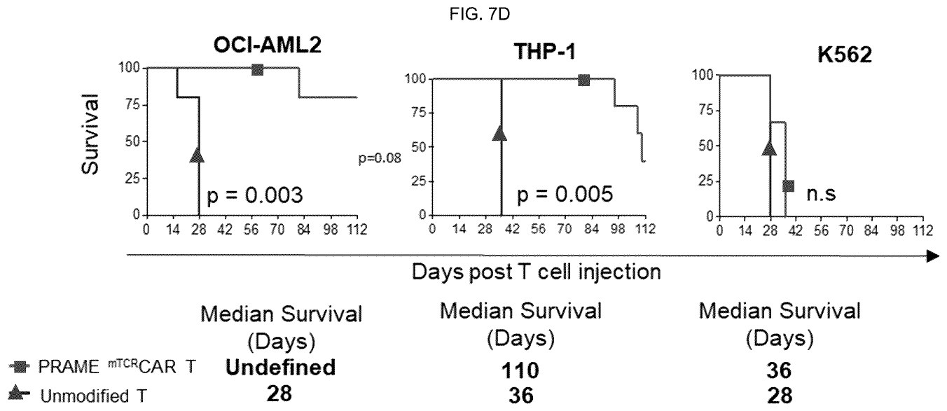

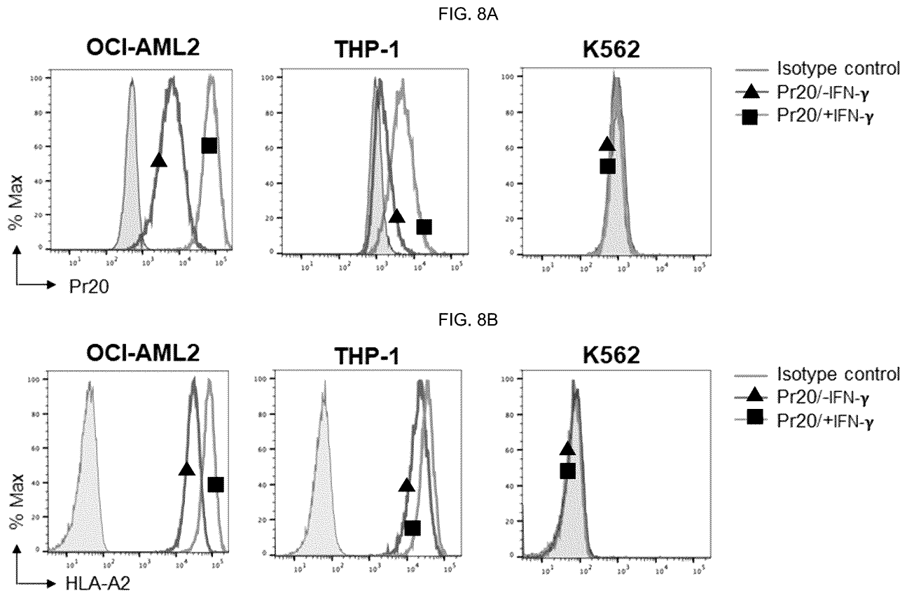

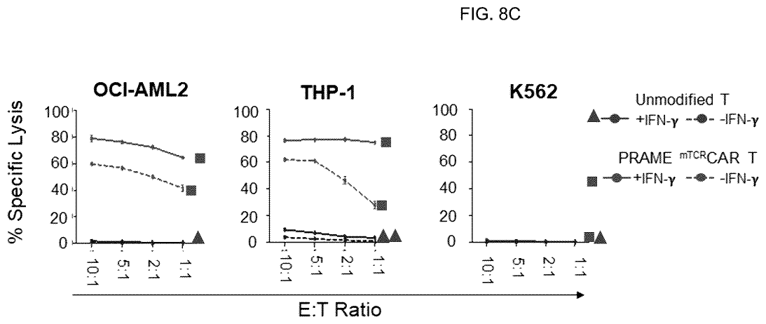

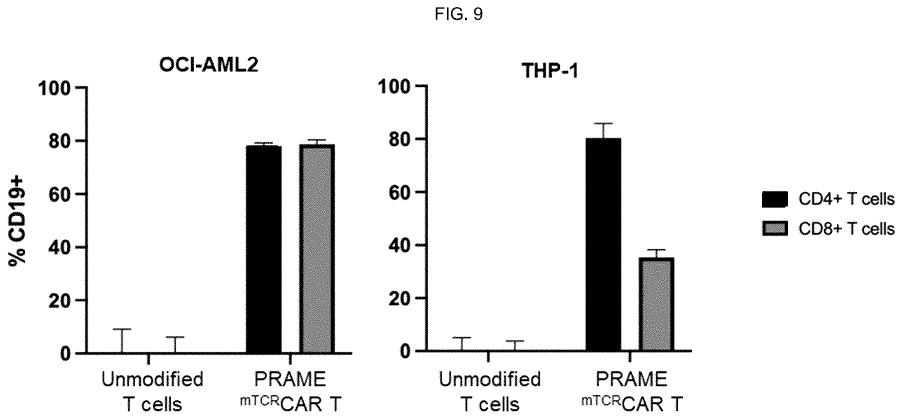

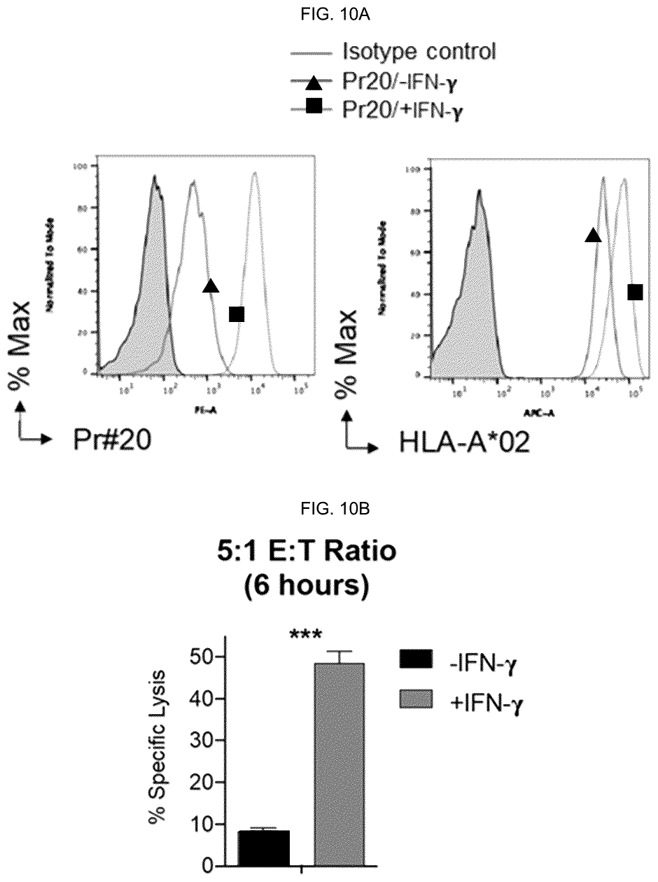

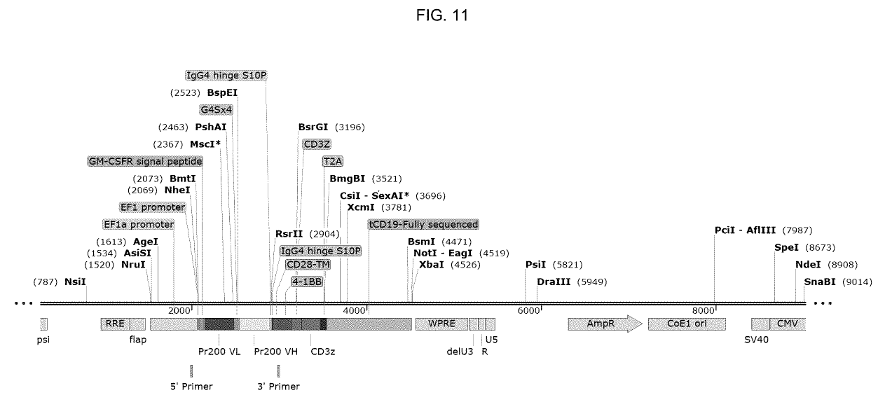

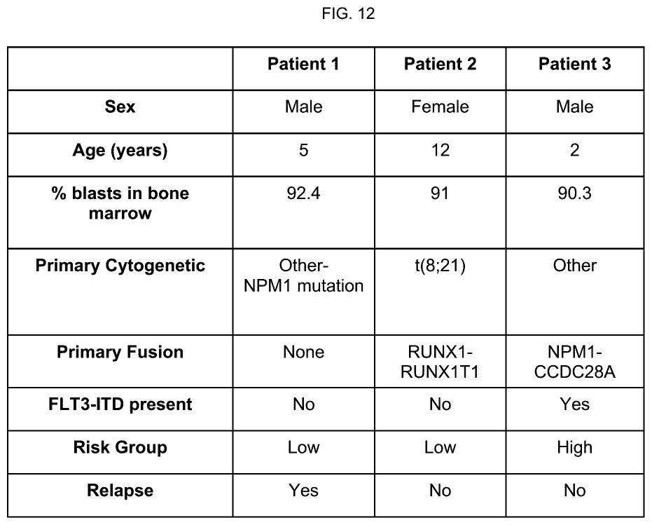

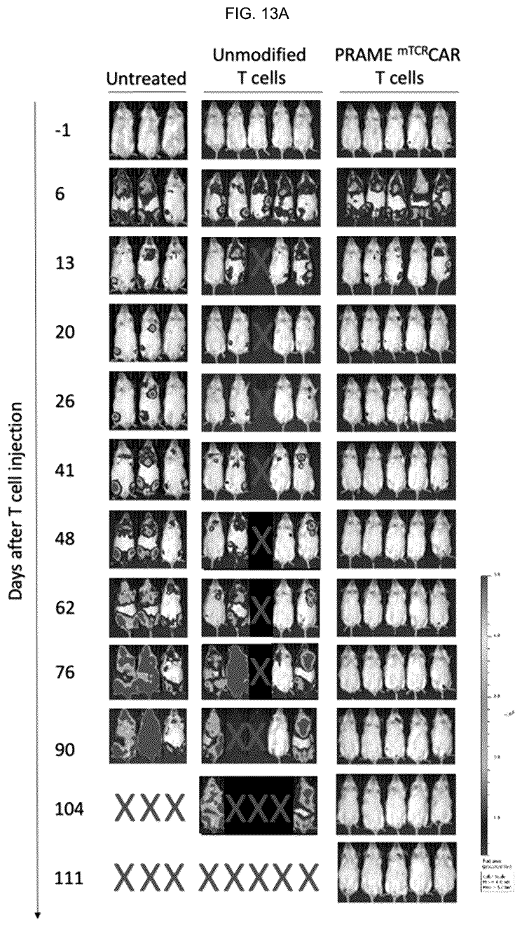

PRAME ^{mTCR}CAR T cells demonstrate potent leukemia clearance and improved survival in vivo. To evaluate the in vivo efficacy of the PRAME ^{mTCR}CAR T cells, human leukemia xenograft models were generated by injecting NSG mice with OCI-AML2, THP-1, and K562 cells transduced with a luciferase expression construct at 1×10⁶ cells per mouse. Following one week after leukemia injection, the leukemia-bearing mice were treated with unmodified or PRAME ^{mTCR}CAR T cells at 5×10⁶ T cells per mouse with 1:1 ratio of CD4 and CD8 T cells. Leukemia burden was monitored by bioluminescence (IVIS) imaging. Treatment with PRAME ^{mTCR}CAR T cells led to leukemia clearance in OCI-AML2-bearing mice, which remained disease free after CAR T cell injection for the entire duration of the study (FIGS. 7A and 7B, left). In contrast, OCI-AML2-bearing mice treated with unmodified T cells exhibited disease progression that led to symptomatic leukemia. The PRAME ^{mTCR}CAR T cells significantly reduced the growth of THP-1 cells but did not completely eradicate the leukemia in vivo (FIGS. 7A and 7B, middle). As expected, treatment with PRAME ^{mTCR}CAR T cell did not affect leukemia growth in K562 xenografts (FIGS. 7A and 7B, right). Consistent with robust anti-leukemia activity of PRAME ^{mTCR}CAR T cells in OCI-AML2-bearing mice, significant expansion of the CAR T cells in the peripheral blood compared to unmodified T cells at day 6 post T cell injection (FIG. 7C, left) was observed. There was no difference in T cell engraftment between unmodified and CAR T cells in THP-1 and K562-bearing mice (FIG. 7C, middle and right, respectively). It was confirmed that the CAR T cells that engrafted in the OCI-AML2 xenografts expressed the transduction marker, truncated CD19 (FIG. 8). Importantly, the anti-leukemia activity of PRAME ^{mTCR}CAR T cells led to a significant increase in survival for OCI-AML2 (p=0.003) and THP-1 (p=0.005), but not K562 xenografts (FIG. 7D). Together, these results demonstrate the in vivo efficacy of PRAME ^{mTCR}CAR T cells against PRAME⁺/HLA-A2⁺ but not PRAME⁺/HLA-A2⁻ leukemias.

IFN-γ treatment increased PRAME/HLA-A2 antigen expression and cytolytic activity of PRAME ^{mTCR}CAR T cells. IFN-γ enhances presentation of tumor-associated antigens by upregulating the immunoproteosome and catalyzing nondestructive cleavage, thereby enhancing MHC peptide surface expression leading to increased T-cell recognition and cytotoxicity (Chang et al., J Clin Invest. 2017, 127(9):3557; Patel et al., Nature. 2017, 548(7669):537-542; Ayers et al., J Clin Invest. 2017, 127(8):2930-2940; Gao et al., Cell. 2016, 167(2):397-404.e9; Shankaran et al., Nature. 2001, 410(6832):1107-1111; and Zhang et al., Cancer Immunol Res. 2019, 7(8):1237-1243). To determine whether IFN-γ enhances PRAME antigen expression, OCI-AML2, THP-1 and K562 cells were treated with IFN-γ for 72 hours and PRAME and HLA-A2 expression was assessed by flow cytometry. As anticipated, treatment with IFN-γ resulted in increased levels of both HLA-A2 and PRAME on OCI-AML2 and THP-1 cells (FIG. 8A, left and middle, respectively). IFN-γ treatment did not affect PRAME and HLA-A2 expression in K562 cells (FIG. 8A, right). Given that IFN-γ enhanced PRAME expression, it was next investigated whether OCI-AML2 and THP-1 cells pre-treated with IFN-γ would be more susceptible to cytotoxicity of PRAME ^{mTCR}CAR T cells. OCI-AML2 and THP-1 cells were incubated with IFN-γ for 72 hours, washed and then co-incubated them with unmodified or PRAME ^{mTCR}CAR T cells at various E:T ratios. T cell killing was assayed after 16 hours of co-incubation. Pre-treatment of target cells with IFN-γ resulted in enhanced cytolytic activity of PRAME ^{mTCR}CAR T cells (FIG. 8B, left and middle). This enhanced activity is dependent on PRAME antigen expression as no significant activity was detected in unmodified T cells co-incubated with THP-1 and OCI-AML2 that were either untreated or pre-treated with IFN-γ. Consistent with target specificity, K562 cells either untreated or pre-treated with IFN-γ were not sensitive to the cytolytic activity of PRAME ^{mTCR}CAR T cells (FIG. 8B, right).

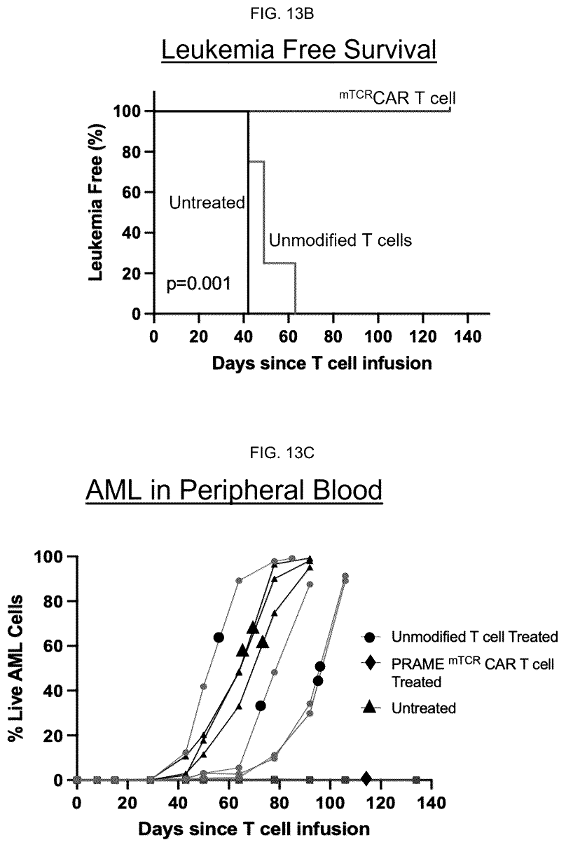

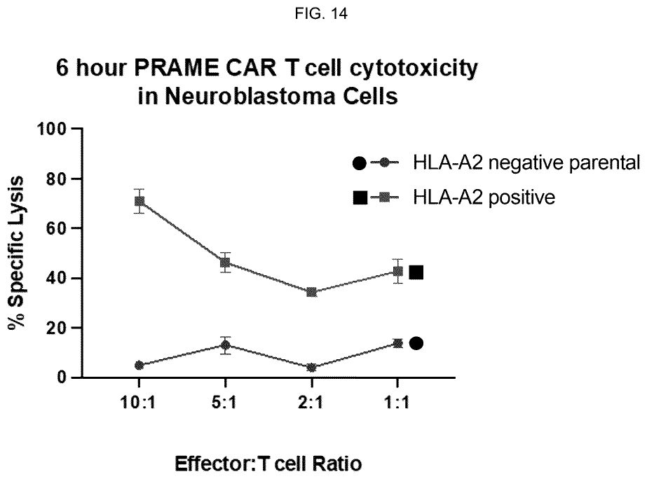

In vitro cytotoxicity in neuroblastoma following treatment with PRAME TCR mimic CAR T cells. At 6 hours of co-incubation with various E:T ratios of neuroblastoma cells and CD8+ PRAME TCR mimic CAR T cells, significant specific cell death was seen. At 24 hours, 98% cell death was seen at all E:T ratios (FIG. 13).

Discussion. Identification of targets whose expression is limited to AML leukemic cells and silent in normal hematopoiesis provides an opportunity for effective therapies with limited to no hematopoietic toxicity. Through comprehensive analysis of the AML transcriptome from over 2000 patients, PRAME as one such AML-restricted target with no expression in normal hematopoiesis was identified. In this Example, the intracellular protein PRAME was prioritized as a target for modified CAR T development based on a TCR mimic antibody (Pr20) previously developed by Chang et al, which has been reported to have high binding affinity (4-5 nM K_{D}) (Chang et al., J Clin Invest. 2017, 127(9):3557). The Pr20 mAb that recognizes PRAME ALY peptide (SEQ ID NO: 94) in complex with HLA-A2 was used, and the specificity of the Pr20 antibody against PRAME+/HLA-A2+ leukemias was confirmed. It was further demonstrated that Pr20 recognizes PRAME/HLA-A2 in primary AML blasts. Using this antibody sequence, PRAME ^{mTCR}CAR T cells were developed against AML cells expressing PRAME. PRAME ^{mTCR}CAR T cells exhibit preclinical efficacy in eliminating AML cells in vitro and in vivo. This work highlights the therapeutic potential of targeting PRAME in AML and provides a novel approach to target intracellular antigens with CAR T cells.

PRAME represents a promising target for immunotherapy as its expression is limited to the reproductive tissues (Al-Khadairi et al., 2019, 11(7):984; Chang et al., J Clin Invest. 2017, 127(9):3557; and Xu et al., Cell Prolif. 2020, 53(3):e12770), and is broadly expressed in many cancers, including AML (Wadelin et al., Mol Cancer. 2010, 9:226; Ikeda et al., Immunity. 1997, 6(2):199-208; Epping et al., Cancer Res. 2006, 6(22):10639-10642; Oberthuer et al., Clin Cancer Res. 2004, 10(13):4307-4313; Tajeddine et al., Cancer Res. 2005, 65(16):7348-7355; 17; Thongprasert et al., Lung Cancer. 2016, 101:137-144; Bankovic et al., Lung Cancer. 2010, 67(2):151-159; Pan et al., Asia Pac J Clin Oncol. 2017, 13(5):e212-e223; Greiner et al., Int J Cancer. 2004, 108(5):704-711; Ding et al., Cancer Biol Med. 2012, 9(1):73-76; and Radich et al., Proc Natl Acad Sci USA. 2006, 103(8):2794-2799). In this Example, it is shown that PRAME is expressed in a substantial number of pediatric and adult AML. HLA-A2 is the most common HLA-1 subtype, found in 47% of pediatric patients with AML, thus targeting HLA-A2⁺ patients will benefit a substantial portion of patients. PRAME and HLA-A2 are regulatable pharmacologically (Chang et al., J Clin Invest. 2017, 127(9):3557; Bourne et al., Blood Adv. 2022, 6(14):4107-4121; Oh et al., Cancer Immunol Res. 2019, 7(12):1984-1997; and Brea et al., Cancer Immunol Res. 2016, 4(11):936-947), therefore future work might involve upregulating the epitope with a combination therapy. Interestingly, PRAME expression is enriched in t(8;21) AML, where nearly all of t(8;21) leukemias express PRAME suggesting that the fusion protein AML1/ETO may directly or indirectly promote PRAME expression in this leukemia. Importantly, PRAME transcript expression was not detected in normal peripheral blood CD34+ and bone marrow samples suggesting that targeting PRAME would not impact normal hematopoiesis.

Given the successes of CAR T cells in treating B-cell malignancies, PRAME ^{mTCR}CAR T cells binding the Pr20 antigen formed by the PRAME ALY peptide (SEQ ID NO: 94):HLA-A2 complex were developed. Although PRAME ^{mTCR}CAR T cells completely eradicated OCI-AML2 leukemia in vivo, they were less effective against THP-1 leukemia. Without being bound by theory, it is hypothesized this is due to lower antigen density in THP-1 cells [MFI (fold change to isotype control): 2.75 fold (THP-1) versus 5 fold (OCI-AML2)], though antigen density was not directly studied. In contrast to OCI-AML2 cells, with T cell expansion noted on Day 6, it is hypothesized that the lower antigen density on THP-1 cells led to a delay in T cell expansion, but ongoing exposure of PRAME-expressing cells resulted in sufficient T cell activation leading to reduction in leukemia burden and prolonged survival. Strategies that enhance PRAME antigen expression would result in increased efficacy of PRAME ^{mTCR}CAR T cells. IFN-γ treatment increases expression of the PRAME ALY(SEQ ID NO: 94)/HLA-A2 complex by inducing nondestructive cleavage sites by the immunoproteosome (Chang et al., J Clin Invest. 2017, 127(9):3557). Here, it is demonstrated that treatment with IFN-γ resulted in increased expression of PRAME/HLA-A2 leading to improved cytolytic activity of PRAME ^{mTCR}CAR T cells against OCI-AML2 and THP-1 cells. IFN-γ has been shown to be well-tolerated in prior clinical trials as a monotherapy and in combination with chemotherapy (Alberts et al., Gynecol Oncol. 2008, 109(2):174-181; Giannopoulos et al., Clin Cancer Res. 2003, 9(15):5550-5558; and Windbichler et al., Br J Cancer. 2000, 82(6):1138-1144). It is proposed that IFN-γ provides a useful strategy to increase efficacy of PRAME ^{mTCR}CAR T cells and should be evaluated in future PRAME ^{mTCR}CAR T cell therapy clinical trials for AML. In addition, incorporating different CAR elements such as scFv binding and signaling components (i.e CD28z) can increase antileukemic sensitivity and efficacy (Srivastava and Riddell. Trends Immunol. 2015; 36(8):494-502), especially against AML cells with low antigen density, and should be evaluated to optimize the efficacy of PRAME ^{mTCR}CAR T cells.

The therapeutic potential of targeting PRAME with ^{mTCR}CAR T cells in AML has been demonstrated. A potent, target-specific reactivity of PRAME ^{mTCR}CAR T cells against PRAME⁺/HLA-A2⁺ AML cell lines, but not PRAME⁺/HLA-A2⁻ K562 cell line both in vitro and in vivo is shown. Additionally, the target-specific cytolytic activity of PRAME ^{mTCR}CAR T cells against PRAME⁺/HLA-A2⁺ primary AML blasts is demonstrated. The results presented provide a novel approach to target PRAME with ^{mTCR}CAR T cells and compelling data supporting use of PRAME ^{mTCR}CAR T cells to treat AML.

### (x) CLOSING PARAGRAPHS

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

To the extent not explicitly provided herein, coding sequences for proteins disclosed herein and protein sequences for coding sequences disclosed herein can be readily derived from one of ordinary skill in the art.

Variants of the sequences disclosed and referenced herein are also included. Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular signaling components transmit activation or inhibition signals comparable to a wild-type reference when in the activated state of the current disclosure. Functional variants and functional fragments of binding domains bind their cognate antigen or ligand at a level comparable to a wild-type reference.

Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein.

In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Binds” refers to an association of a binding domain (of, for example, a CAR binding domain or an antibody binding domain) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N. Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability of a CAR-expressing cell disclosed herein to kill PRAME ALY(SEQ ID NO: 94)/HLA-A2-expressing cells, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A chimeric antigen receptor (CAR) that, when expressed by a cell, comprises an extracellular component linked to an intracellular component through a transmembrane domain, wherein the extracellular component comprises a Preferentially Expressed Antigen in Melanoma (PRAME) ALY(SEQ ID NO: 94)/HLA-A2 binding domain and the intracellular component comprises an effector domain.

2. (canceled)

3. The CAR of claim 1, wherein the binding domain comprises a single chain variable fragment (scFv) having a sequence as set forth in SEQ ID NO: 43 or SEQ ID NO: 44 or a sequence having at least 90% sequence identity to the sequence as set forth in SEQ ID NO: 43 and/or SEQ ID NO: 44.

4. (canceled)

5. The CAR of claim 1, wherein the binding domain comprises a single chain variable fragment (scFv) encoded by a sequence as set forth in SEQ ID NO: 3 or SEQ ID NO: 4 or a sequence having at least 90% sequence identity to the sequence as set forth in SEQ ID NO: 3 and or SEQ ID NO: 4.

6. (canceled)

7. The CAR of claim 1, wherein the binding domain comprises a variable light chain comprising a sequence as set forth in SEQ ID NO: 41 or a sequence having at least 90% sequence identity to the sequence as set forth in SEQ ID NO: 41 and a variable heavy chain comprising a sequence as set forth in SEQ ID NO: 42 or a sequence having at least 90% sequence identity to the sequence as set forth in SEQ ID NO: 42.

8. (canceled)

9. The CAR of claim 1, wherein the binding domain comprises a variable light chain encoded by a sequence as set forth in SEQ ID NO: 1 or a sequence having at least 90% sequence identity to the sequence as set forth in SEQ ID NO: 1 and a variable heavy chain encoded by a sequence as set forth in SEQ ID NO: 2 or a sequence having at least 90% sequence identity to the sequence as set forth in SEQ ID NO: 2.

10. (canceled)

11. The CAR of claim 1, wherein the binding domain comprises a variable light chain complementarity determining region (CDRL) 1 as set forth in SEQ ID NO: 48, a CDRL2 as set forth in SEQ ID NO: 49, and a CDRL3 as set forth in SEQ ID NO: 50 and a variable heavy chain with complementarity determining regions (CDRH) 1 as set forth in SEQ ID NO: 45, a CDRH2 as set forth in SEQ ID NO: 46, and a CDRH3 as set forth in SEQ ID NO: 47;
a CDRL1 as set forth in SEQ ID NO: 48, a CDRL2 as set forth in SEQ ID NO: 49, a CDRL3 as set forth in SEQ ID NO: 50, a CDRH1 as set forth in SEQ ID NO: 51, a CDRH2 as set forth in SEQ ID NO: 52, and a CDRH3 as set forth in SEQ ID NO: 53;
a CDRL1 as set forth in SEQ ID NO: 56, a CDRL2 including the sequence SNN, a CDRL3 as set forth in SEQ ID NO: 50, a CDRH1 as set forth in SEQ ID NO: 54, a CDRH2 as set forth in SEQ ID NO: 55, and a CDRH3 as set forth in SEQ ID NO: 47;
a CDRL1 as set forth in SEQ ID NO: 48, a CDRL2 as set forth in SEQ ID NO: 58, a CDRL3 as set forth in SEQ ID NO: 50, a CDRH1 as set forth in SEQ ID NO: 54, a CDRH2 as set forth in SEQ ID NO: 57, and a CDRH3 as set forth in SEQ ID NO: 53; or
a CDRL1 as set forth in SEQ ID NO: 62, a CDRL2 as set forth in SEQ ID NO: 63, a CDRL3 as set forth in SEQ ID NO: 64, a CDRH1 as set forth in SEQ ID NO: 59, a CDRH2 as set forth in SEQ ID NO: 60, and a CDRH3 as set forth in SEQ ID NO: 61.

12. The CAR of claim 1, wherein the extracellular component further comprises a spacer region encoded by a sequence as set forth in SEQ ID NO: 16 or 17 or a sequence having at least 90% sequence identity to the sequence as set forth in SEQ ID NO: 16 and/or SEQ ID NO: 17.

13-16. (canceled)

17. The CAR of claim 1, wherein the intracellular effector domain comprises all or a portion of the signaling domain of CD3ζ and 4-11BB.

18-25. (canceled)

26. The CAR of claim 1, wherein the transmembrane domain comprises a CD28 transmembrane domain.

27-30. (canceled)

31. The CAR of claim 1, further comprising a control feature selected from a tag cassette, a transduction marker, and/or a suicide switch.

32-39. (canceled)

40. The CAR of claim 1, comprising a sequence as set forth in SEQ ID NO: 5, SEQ ID NO: 7, or SEQ ID NO: 9 or a sequence having at least 90% sequence identity to the sequence as set forth in SEQ ID NO: 5, SEQ ID NO: 7, and/or SEQ ID NO: 9.

41. (canceled)

42. The CAR of claim 1, encoded by a sequence as set forth in SEQ ID NO: 6, SEQ ID NO: 8, SEQ ID NO: 10, or SEQ ID NO: 11 or a sequence having at least 90% sequence identity to the sequence as set forth in SEQ ID NO: 6, SEQ ID NO: 8, SEQ ID NO: 10, and/or SEQ ID NO: 11.

43. (canceled)

44. A genetic construct encoding the CAR of claim 1.

45. The genetic construct of claim 44, comprising a sequence as set forth in SEQ ID NO: 11 or a sequence having at least 90% sequence identity to the sequence as set forth in SEQ ID NO: 11.

46-57. (canceled)

58. A method of treating a subject with a PRAME ALY (SEQ ID NO:94)/HLA-A2 expressing cancer, the method comprising administering a therapeutically effective amount of the CAR of claim 1 to the subject thereby treating the subject with the PRAME ALY(SEQ ID NO: 94)/HLA-A2 expressing cancer.

59. The method of claim 58, wherein the PRAME ALY(SEQ ID NO: 94)/HLA-A2 expressing cancer is acute myeloid leukemia (AML).

60. The method of claim 59, wherein the AML is t(8;21) AML, Inv(16) AML, or KMT2A-r AML.

61. The method of claim 58, further comprising screening the subject for the PRAME ALY/HLA-A2 expressing cancer.

62. The method of claim 61, further comprising selecting the subject for treatment based on the screening.

63. The method of claim 58, further comprising administering interferon gamma (IFNγ) to the subject.

64. (canceled)


SEQUENCE LISTING (94 sequences)

SEQ ID NO: 1 - DNA, 336 bp, synthetic construct
  source 1..336: mol_type = other DNA; note = Pr20 VL encoding sequence
caggctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttcttatgtcttcggaactgggaccaaggtcaccgtcctaggt

SEQ ID NO: 2 - DNA, 351 bp, synthetic construct
  source 1..351: mol_type = other DNA; note = Pr20 Vh encoding sequence
caggtgcagctggtgcaatctggagctgaggtgaggaagcctggggcctcagtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacactgccgtgtattactgtgcgcgccattacggtcagtggtgggattactggggtcaaggtactctggtgaccgtctcctca

SEQ ID NO: 3 - DNA, 747 bp, synthetic construct
  source 1..747: mol_type = other DNA; note = Pr20 VL-VH scFv encoding sequence
caggctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttcttatgtcttcggaactgggaccaaggtcaccgtcctaggtggcggcggaggaagcggaggcggaggatctggcggagggggctccggagggggaggctctcaggtgcagctggtgcaatctggagctgaggtgaggaagcctggggcctcagtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacactgccgtgtattactgtgcgcgccattacggtcagtggtgggattactggggtcaaggtactctggtgaccgtctcctca

SEQ ID NO: 4 - DNA, 747 bp, synthetic construct
  source 1..747: mol_type = other DNA; note = Pr20 VH-VL scFv encoding sequence
caggtgcagctggtgcaatctggagctgaggtgaggaagcctggggcctcagtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacactgccgtgtattactgtgcgcgccattacggtcagtggtgggattactggggtcaaggtactctggtgaccgtctcctcaggcggcggaggaagcggaggcggaggatctggcggagggggctccggagggggaggctctcaggctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttcttatgtcttcggaactgggaccaaggtcaccgtcctaggt

SEQ ID NO: 5 - protein, 443 aa, synthetic construct
  source 1..443: note = CAR Sequence (Construct including Pr200 VL-VH scFv, IgG4 hinge, CD28 TM, 4-1BB Intracellular Domain, and CD3zeta Intracellular Domain)
QAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGSYVFGTGTKVTVLGGGGGSGGGGSGGGGSGGGGSQVQLVQSGAEVRKPGASVKVSCKASGGTFSSYAISWVRQAPGQGLEWMGRIIPILGIANYAQKFQGRVTITADKSTSTAYMELSSLRSEDTAVYYCARHYGQWWDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 1329 bp, synthetic construct
  source 1..1329: mol_type = other DNA; note = CAR Encoding Sequence (Construct encoding Pr200 VL-VH scFv, IgG4 hinge, CD28 TM, 4-1BB Intracellular Domain, and CD3zeta Intracellular Domain)
caggctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttcttatgtcttcggaactgggaccaaggtcaccgtcctaggtggcggcggaggaagcggaggcggaggatctggcggagggggctccggagggggaggctctcaggtgcagctggtgcaatctggagctgaggtgaggaagcctggggcctcagtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacactgccgtgtattactgtgcgcgccattacggtcagtggtgggattactggggtcaaggtactctggtgaccgtctcctcagagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 7 - protein, 465 aa, synthetic construct
  source 1..465: note = CAR Sequence with Signal Peptide (Construct including GM-CSFR signal peptide, Pr200 VL-VH scFv, IgG4 hinge, CD28 TM, 4-1BB Intracellular Domain, and CD3zeta Intracellular Domain)
MLLLVTSLLLCELPHPAFLLIPQAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGSYVFGTGTKVTVLGGGGGSGGGGSGGGGSGGGGSQVQLVQSGAEVRKPGASVKVSCKASGGTFSSYAISWVRQAPGQGLEWMGRIIPILGIANYAQKFQGRVTITADKSTSTAYMELSSLRSEDTAVYYCARHYGQWWDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - DNA, 1395 bp, synthetic construct
  source 1..1395: mol_type = other DNA; note = CAR Encoding Sequence including Signal Peptide (Construct encoding GM-CSFR signal peptide, Pr200 VL-VH scFv, IgG4 hinge, CD28 TM, 4-1BB Intracellular Domain, and CD3zeta Intracellular Domain)
atgctgctgctcgtgaccagcctgctgctgtgcgaactgccccaccctgcctttctgctgatcccccaggctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttcttatgtcttcggaactgggaccaaggtcaccgtcctaggtggcggcggaggaagcggaggcggaggatctggcggagggggctccggagggggaggctctcaggtgcagctggtgcaatctggagctgaggtgaggaagcctggggcctcagtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacactgccgtgtattactgtgcgcgccattacggtcagtggtgggattactggggtcaaggtactctggtgaccgtctcctcagagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 9 - protein, 812 aa, synthetic construct
  source 1..812: note = CAR Sequence including Signal Peptide and Transduction Marker (Construct including GM-CSFR signal peptide, Pr200 VL-VH scFv, IgG4 hinge, CD28 TM, 4-1BB Intracellular Domain, CD3zeta Intracellular Domain, T2A, and tCD19)
MLLLVTSLLLCELPHPAFLLIPQAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGSYVFGTGTKVTVLGGGGGSGGGGSGGGGSGGGGSQVQLVQSGAEVRKPGASVKVSCKASGGTFSSYAISWVRQAPGQGLEWMGRIIPILGIANYAQKFQGRVTITADKSTSTAYMELSSLRSEDTAVYYCARHYGQWWDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 10 - DNA, 2439 bp, synthetic construct
  source 1..2439: mol_type = other DNA; note = CAR Encoding Sequence including Signal Peptide and Transduction Marker (Construct encoding GM-CSFR signal peptide, Pr200 VL-VH scFv, IgG4 hinge, CD28 TM, 4-1BB Intracellular Domain, CD3zeta Intracellular Domain, T2A, and tCD19)
atgctgctgctcgtgaccagcctgctgctgtgcgaactgccccaccctgcctttctgctgatcccccaggctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttcttatgtcttcggaactgggaccaaggtcaccgtcctaggtggcggcggaggaagcggaggcggaggatctggcggagggggctccggagggggaggctctcaggtgcagctggtgcaatctggagctgaggtgaggaagcctggggcctcagtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacactgccgtgtattactgtgcgcgccattacggtcagtggtgggattactggggtcaaggtactctggtgaccgtctcctcagagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 11 - DNA, 9287 bp, synthetic construct
  source 1..9287: mol_type = other DNA; note = Genetic Construct (Construct including/encoding psi, RRE, Ef1a promoter, GM-CSFR signal peptide, Pr200 VL-VH scFv, IgG4 hinge, CD28 TM, 4-1BB Intracellular Domain, CD3zeta Intracellular Domain, T2A, tCD19, WPRE, delU3, R, U5, AmpR, CoE1 ori, SV40, and CMV)
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatcccccaggctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttcttatgtcttcggaactgggaccaaggtcaccgtcctaggtggcggcggaggaagcggaggcggaggatctggcggagggggctccggagggggaggctctcaggtgcagctggtgcaatctggagctgaggtgaggaagcctggggcctcagtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacactgccgtgtattactgtgcgcgccattacggtcagtggtgggattactggggtcaaggtactctggtgaccgtctcctcagagtctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 12 - DNA, 60 bp, synthetic construct
  source 1..60: mol_type = other DNA; note = G4Sx4 linker encoding sequence
ggcggcggaggaagcggaggcggaggatctggcggagggggctccggagggggaggctct

SEQ ID NO: 13 - protein, 22 aa, synthetic construct
  source 1..22: note = GM-CSFR Signal Peptide
MLLLVTSLLLCELPHPAFLLIP

SEQ ID NO: 14 - DNA, 66 bp, synthetic construct
  source 1..66: mol_type = other DNA; note = GM-CSFR Signal Peptide encoding sequence
atgctgctgctcgtgaccagcctgctgctgtgcgaactgccccaccctgcctttctgctgatcccc

SEQ ID NO: 15 - protein, 12 aa, synthetic construct
  source 1..12: note = IgG4 hinge sequence-A (IgG4 hinge S10P or short spacer)
ESKYGPPCPPCP

SEQ ID NO: 16 - DNA, 36 bp, synthetic construct
  source 1..36: mol_type = other DNA; note = IgG4 hinge coding sequence-A (IgG4 hinge S10P or short spacer)
gagtctaagtacggaccgccctgccccccttgccct

SEQ ID NO: 17 - DNA, 36 bp, synthetic construct
  source 1..36: mol_type = other DNA; note = IgG4 hinge coding sequence-B (short spacer)
gagtctaagtacggaccgccttgcccaccgtgccca

SEQ ID NO: 18 - DNA, 321 bp, synthetic construct
  source 1..321: mol_type = other DNA; note = Spacer coding sequence (IgG4-int(DS))
ggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 19 - DNA, 648 bp, synthetic construct
  source 1..648: mol_type = other DNA; note = Spacer coding sequence (IgG4-long)
gcaccacctgtggcaggaccgtcagtcttcctcttcccaccaaaacccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccaaagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 20 - protein, 112 aa, synthetic construct
  source 1..112: note = CD3Zeta protein-A
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 114 aa, synthetic construct
  source 1..114: note = CD3Zeta protein-B
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 336 bp, synthetic construct
  source 1..336: mol_type = other DNA; note = CD3Zeta coding sequence
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 23 - protein, 43 aa, synthetic construct
  source 1..43: note = 4-1BB protein-A
VKRGRKKLLYIFKQPFMRPVQTTQEEDGCSCRFPEEEEGGCEL

SEQ ID NO: 24 - protein, 41 aa, synthetic construct
  source 1..41: note = 4-1BB protein-B
VKRGRKKLLYIFKQPFMRPVQTTQEEDGCSCRFPEEEEGGC

SEQ ID NO: 25 - DNA, 129 bp, synthetic construct
  source 1..129: mol_type = other DNA; note = 4-1BB signaling coding sequence-A
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 26 - DNA, 126 bp, synthetic construct
  source 1..126: mol_type = other DNA; note = 4-1BB signaling coding sequence-B
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 27 - protein, 27 aa, synthetic construct
  source 1..27: note = CD28TM protein-A
MFWVLVVVGGVLACYSLLVTVAFIIFW

SEQ ID NO: 28 - protein, 26 aa, synthetic construct
  source 1..26: note = CD28TM protein-B
FWVLVVVGGVLACYSLLVTVAFIIFW

SEQ ID NO: 29 - protein, 81 aa, synthetic construct
  source 1..81: note = CD28TM coding sequence-A
ATGTTCTGGGTGCTGGTGGTGGTCGGAGGCGTGCTGGCCTGCTACAGCCTGCTGGTCACCGTGGCCTTCATCATCTTTTGG

SEQ ID NO: 30 - DNA, 84 bp, synthetic construct
  source 1..84: mol_type = other DNA; note = CD28TM coding sequence-B
atgttctgggtgctggtggtggtcggaggcgtgctggcctgctacagcctgctggtcaccgtggccttcatcatcttttgggtg

SEQ ID NO: 31 - DNA, 81 bp, synthetic construct
  source 1..81: mol_type = other DNA; note = CD28TM coding sequence-C
atgttctgggtgctggtggtggtgggcggggtgctggcctgctacagcctgctggtgacagtggccttcatcatcttttgg

SEQ ID NO: 32 - protein, 24 aa, synthetic construct
  source 1..24: note = T2A Sequence-A
LEGGGEGRGSLLTCGDVEENPGPR

SEQ ID NO: 33 - DNA, 72 bp, synthetic construct
  source 1..72: mol_type = other DNA; note = T2A encoding sequence
ctcgagggcggcggagagggcagaggaagtcttctaacatgcggtgacgtggaggagaatccaggccctagg

SEQ ID NO: 34 - protein, 21 aa, synthetic construct
  source 1..21: note = T2A Sequence-B
GSGEGRGSLLTCGDVEENPGP

SEQ ID NO: 35 - protein, 26 aa, synthetic construct
  source 1..26: note = P2A
GSGATNFSLLKQAGDVEENPGPGASG

SEQ ID NO: 36 - protein, 24 aa, synthetic construct
  source 1..24: note = E2A
GSGQCTNYALLKLAGDVESNPGPP

SEQ ID NO: 37 - protein, 25 aa, synthetic construct
  source 1..25: note = F2A
GSGVKQTLNFDLLKLAGDVESNPGP

SEQ ID NO: 38 - protein, 323 aa, synthetic construct
  source 1..323: note = tCD19
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 39 - DNA, 972 bp, synthetic construct
  source 1..972: mol_type = other DNA; note = tCD19 coding sequence
atgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 40 - protein, 509 aa, Homo sapiens [sequence omitted]

SEQ ID NO: 41 - protein, 112 aa, synthetic construct
  source 1..112: note = variable light chain of the Pr20 antibody
QAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGSYVFGTGTKVTVLG

SEQ ID NO: 42 - protein, 117 aa, synthetic construct
  source 1..117: note = variable heavy chain of the Pr20 antibody
QVQLVQSGAEVRKPGASVKVSCKASGGTFSSYAISWVRQAPGQGLEWMGRIIPILGIANYAQKFQGRVTITADKSTSTAYMELSSLRSEDTAVYYCARHYGQWWDYWGQGTLVTVSS

SEQ ID NO: 43 - protein, 249 aa, synthetic construct
  source 1..249: note = exemplary scFv derived from the Pr20 antibody
QAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGSYVFGTGTKVTVLGGGGGSGGGGSGGGGSGGGGSQVQLVQSGAEVRKPGASVKVSCKASGGTFSSYAISWVRQAPGQGLEWMGRIIPILGIANYAQKFQGRVTITADKSTSTAYMELSSLRSEDTAVYYCARHYGQWWDYWGQGTLVTVSS

SEQ ID NO: 44 - protein, 249 aa, synthetic construct
  source 1..249: note = exemplary scFv derived from the Pr20 antibody
QVQLVQSGAEVRKPGASVKVSCKASGGTFSSYAISWVRQAPGQGLEWMGRIIPILGIANYAQKFQGRVTITADKSTSTAYMELSSLRSEDTAVYYCARHYGQWWDYWGQGTLVTVSSGGGGSGGGGSGGGGSGGGGSQAVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGSYVFGTGTKVTVLG

SEQ ID NO: 45 - protein, 13 aa, synthetic construct
  source 1..13: note = PRAME ALY / HLA-A2 CDR Binding Set Sequence
KASGGTFSSYAIS

SEQ ID NO: 46 - protein, 10 aa, synthetic construct
  source 1..10: note = PRAME ALY / HLA-A2 CDR Binding Set Sequence
RIIPILGIAN

SEQ ID NO: 47 - protein, 10 aa, synthetic construct
  source 1..10: note = PRAME ALY / HLA-A2 CDR Binding Set Sequence
ARHYGQWWDY

SEQ ID NO: 48 - protein, 13 aa, synthetic construct
  source 1..13: note = PRAME ALY / HLA-A2 CDR Binding Set Sequence
SGSSSNIGSNTVN

SEQ ID NO: 49 - protein, 8 aa, synthetic construct
  source 1..8: note = PRAME ALY / HLA-A2 CDR Binding Set Sequence
YSNNQRPS

SEQ ID NO: 50 - protein, 12 aa, synthetic construct
  source 1..12: note = PRAME ALY / HLA-A2 CDR Binding Set Sequence
AAWDDSLNGSYV

SEQ ID NO: 51 - protein, 5 aa, synthetic construct
  source 1..5: note = PRAME ALY / HLA-A2 CDR Binding Set Sequence
SYAIS

SEQ ID NO: 52 - protein, 17 aa, synthetic construct
  source 1..17: note = PRAME ALY / HLA-A2 CDR Binding Set Sequence
RIIPILGIANYAQKFQG

SEQ ID NO: 53 - protein, 8 aa, synthetic construct
  source 1..8: note = PRAME ALY / HLA-A2 CDR Binding Set Sequence
HYGQWWDY

SEQ ID NO: 54 - protein, 8 aa, synthetic construct
  source 1..8: note = PRAME ALY / HLA-A2 CDR Binding Set Sequence
GGTFSSYA

SEQ ID NO: 55 - protein, 8 aa, synthetic construct
  source 1..8: note = PRAME ALY / HLA-A2 CDR Binding Set Sequence
IIPILGIA

SEQ ID NO: 56 - protein, 8 aa, synthetic construct
  source 1..8: note = PRAME ALY / HLA-A2 CDR Binding Set Sequence
SSNIGSNT

SEQ ID NO: 57 - protein, 6 aa, synthetic construct
  source 1..6: note = PRAME ALY / HLA-A2 CDR Binding Set Sequence
IPILGI

SEQ ID NO: 58 - protein, 7 aa, synthetic construct
  source 1..7: note = PRAME ALY / HLA-A2 CDR Binding Set Sequence
SNNQRPS

SEQ ID NO: 59 - protein, 6 aa, synthetic construct
  source 1..6: note = PRAME ALY / HLA-A2 CDR Binding Set Sequence
SSYAIS

SEQ ID NO: 60 - protein, 13 aa, synthetic construct
  source 1..13: note = PRAME ALY / HLA-A2 CDR Binding Set Sequence
WMGRIIPILGIAN

SEQ ID NO: 61 - protein, 9 aa, synthetic construct
  source 1..9: note = PRAME ALY / HLA-A2 CDR Binding Set Sequence
ARHYGQWWD

SEQ ID NO: 62 - protein, 7 aa, synthetic construct
  source 1..7: note = PRAME ALY / HLA-A2 CDR Binding Set Sequence
SNTVNWY

SEQ ID NO: 63 - protein, 10 aa, synthetic construct
  source 1..10: note = PRAME ALY / HLA-A2 CDR Binding Set Sequence
LLIYSNNQRP

SEQ ID NO: 64 - protein, 11 aa, synthetic construct
  source 1..11: note = PRAME ALY / HLA-A2 CDR Binding Set Sequence
AAWDDSLNGSY

SEQ ID NO: 65 - protein, 4 aa, synthetic construct
  source 1..4: note = Gly-Ser linker
GGGS

SEQ ID NO: 66 - protein, 9 aa, synthetic construct
  source 1..9: note = Gly-Ser linker
GGGSGGGGS

SEQ ID NO: 67 - protein, 7 aa, synthetic construct
  source 1..7: note = Gly-Ser linker
GGGSGGS

SEQ ID NO: 68 - protein, 9 aa, synthetic construct
  source 1..9: note = Gly-Ser linker
GGGSGGGGS

SEQ ID NO: 69 - protein, 20 aa, synthetic construct
  source 1..20: note = Gly-Ser linker
GGGGSGGGGSGGGGSGGGGS

SEQ ID NO: 70 - protein, 15 aa, synthetic construct
  source 1..15: note = Gly-Ser linker
GGGGSGGGGSGGGGS

SEQ ID NO: 71 - protein, 10 aa, synthetic construct
  source 1..10: note = Gly-Ser linker
GGGGSGGGGS

SEQ ID NO: 72 - protein, 5 aa, synthetic construct
  source 1..5: note = Gly-Ser linker
GGGGS

SEQ ID NO: 73 - protein, 8 aa, synthetic construct
  source 1..8: note = Gly-Ser linker
GGGSGGGS

SEQ ID NO: 74 - protein, 4 aa, synthetic construct
  source 1..4: note = Gly-Ser linker
GGGS

SEQ ID NO: 75 - protein, 6 aa, synthetic construct
  source 1..6: note = Gly-Ser linker
GGSGGS

SEQ ID NO: 76 - protein, 11 aa, synthetic construct
  source 1..11: note = Gly-Ser linker
GGSGGGSGGSG

SEQ ID NO: 77 - protein, 10 aa, synthetic construct
  source 1..10: note = Gly-Ser linker
GGSGGGSGSG

SEQ ID NO: 78 - protein, 8 aa, synthetic construct
  source 1..8: note = Gly-Ser linker
GGSGGGSG

SEQ ID NO: 79 - protein, 5 aa, synthetic construct
  source 1..5: note = spacer region
EAAAK

SEQ ID NO: 80 - protein, 6 aa, synthetic construct
  source 1..6: note = His tag
HHHHHH

SEQ ID NO: 81 - protein, 8 aa, synthetic construct
  source 1..8: note = Flag tag
DYKDDDDK

SEQ ID NO: 82 - protein, 8 aa, synthetic construct
  source 1..8: note = Xpress tag
DLYDDDDK

SEQ ID NO: 83 - protein, 15 aa, synthetic construct
  source 1..15: note = Avi tag
GLNDIFEAQKIEWHE

SEQ ID NO: 84 - protein, 26 aa, synthetic construct
  source 1..26: note = Calmodulin tag
KRRWKKNFIAVSAANRFKKISSSGAL

SEQ ID NO: 85 - protein, 9 aa, synthetic construct
  source 1..9: note = HA tag
YPYDVPDYA

SEQ ID NO: 86 - protein, 10 aa, synthetic construct
  source 1..10: note = Myc tag
EQKLISEEDL

SEQ ID NO: 87 - protein, 8 aa, synthetic construct
  source 1..8: note = Strep tag
WRHPQFGG

SEQ ID NO: 88 - protein, 8 aa, synthetic construct
  source 1..8: note = STREP tag II
WSHPQFEK

SEQ ID NO: 89 - protein, 13 aa, synthetic construct
  source 1..13: note = Softag 1
SLAELLNAGLGGS

SEQ ID NO: 90 - protein, 8 aa, synthetic construct
  source 1..8: note = Softag 3
TQDPSRVG

SEQ ID NO: 91 - protein, 14 aa, synthetic construct
  source 1..14: note = V5 tag
GKPIPNPLLGLDST

SEQ ID NO: 92 - protein, 275 aa, synthetic construct [sequence omitted]

SEQ ID NO: 93 - protein, 100 aa, synthetic construct [sequence omitted]

SEQ ID NO: 94 - protein, 10 aa, synthetic construct
  source 1..10: note = PRAME peptide
ALYVDSLFFL